(19) United States
(12) Patent Application Publication
(10) Pub. No.: US 2019/0073605 A1
(43) Pub. Date: Mar. 7, 2019

(54) SYSTEMS AND METHODS FOR REAL-TIME NEURAL COMMAND CLASSIFICATION AND TASK EXECUTION

(71) Applicant: Andrew Jay KELLER, Norwalk, CT (US)

(72) Inventor: Andrew Jay KELLER, Norwalk, CT (US)

(21) Appl. No.: 15/907,268

(22) Filed: Feb. 27, 2018

Related U.S. Application Data
(60) Provisional application No. 62/464,112, filed on Feb. 27, 2017.

(51) Int. Cl. G06N 7/00; A61B 5/0478; A61B 5/00; G06K 9/00

(52) U.S. Cl. CPC G06N 7/005; A61B 5/0478; G06K 9/00536; A61B 5/7267; A61B 5/6814

(57) ABSTRACT

A biosignal acquisition system for transmitting context commands based on sensed biosignals is disclosed. The biosignal acquisition system may include a computer, a biosensor and a biosignal acquisition device. The biosensor may be configured to be in contact with a scalp of an individual. The biosignal acquisition device may be operably coupled to the biosensor and configured to amplify signals received by the biosensor and communicate the amplified signal to the computer. The computer may be configured to receive the amplified signals from the biosignal acquisition device, generate a feature matrix based on the amplified signals, identify a brain switch associated with each feature in the feature matrix, predict the probability that a classifier is associated with the brain switch of each feature, and fit the classifier based on the feature matrix.

### CROSS-REFERENCE TO RELATED APPLICATION

The present application claims the benefit of, and priority to, U.S. Provisional Patent Application No. 62/464,112 filed on Feb. 27, 2017, entitled “SYSTEM FOR REALTIME NEUTRAL COMMAND CLASSIFICATION AND INTERCHANGEABLE TASK EXECUTION,” the entire contents of which are hereby incorporated by reference in their entirety.

### BACKGROUND

### Technical Field

The present disclosure is directed to systems and methods for acquiring and analyzing biosignals. More particularly, the present disclosure relates to systems and methods of collecting, analyzing, and transmitting neural commands via a computing device.

### Description of Related Art

Existing bioelectrical signal (herein referred to as “biosignal”) collection systems are often associated with Electrocardiograph (ECG), Electromyography (EMG), Electroencephalogram (EEG), and Electrooculogram (EOG) devices (herein referred to as biosignal collection devices). Such biosignal collection devices are typically used to scan and collect biosignals when diagnosing disease, studying brainwave patterns, and so on. Existing devices, such as biosignal acquisition headsets, are often bulky and reserved for use by academics and researchers.

Researchers have proposed collecting biosignal data in a centralized database to remotely store the biosignal data when collected from multiple users. However, since most biosignal acquisition occurs in a lab or research facility, the available data for storage and analysis may be limited and/or not representative of typical biosignals. For example, researchers often focus on helping partially or completely paralyzed individual 101s (or accessibility challenged individual 101s) to interact with their surrounding environment where such interaction would otherwise be impossible (i.e., would not be possible through physical therapy, etc.). These individual 101s need a high accuracy, real-time, communication systems made possible through an invasive electrode array placed physically on top of the brain of the individual 101. While such systems may be functional, such function comes at significant cost and potential medical risk. As such, less invasive systems and methods of collecting and analyzing biosignals, and making use of such biosignals, are desired.

### SUMMARY

According to an embodiment, a biosignal acquisition system for transmitting context commands based on sensed biosignals is disclosed. The system may include a computer, a biosensor, and a biosignal acquisition device. The biosensor may be configured to be in contact with a scalp of an individual. The biosignal acquisition device may be operably coupled to the biosensor and configured to amplify signals received by the biosensor and communicate the amplified signal to the computer. The computer may include a processor and memory having instructions which, when executed by the processor, cause the computer to, receive the amplified signals from the biosignal acquisition device, generate a feature matrix based on the amplified signals, identify a brain switch associated with each feature in the feature matrix, predict a probability that a classifier is associated with the brain switch of each feature, and fit the classifier based on the feature matrix.

In aspects, the memory may have further instructions stored thereon which, when executed by the processor, cause the computer to initialize a weight vector having a weight associated with the classifier. The weight may correspond to a probability that the classifier is reliable.

According to aspects, additional signals may be received from the biosensors and amplified by the biosignal acquisition device prior to being transmitted to the computer.

In aspects, the memory of the computer may have further instructions stored thereon which, when executed by the processor, cause the computer to generate a second feature matrix based on the additional amplified signals, identify the brain switch associated with each feature of the second feature matrix, predict the probability that a real-time classifier is associated with the brain switch of each feature, fit the real-time classifier based on the second feature matrix, and refit the classifier based on the fitting of the real-time classifier.

According to aspects, refitting the classifier may include adding a weight of the real-time classifier to the weight vector.

In aspects, the weight of the real-time classifier stored in the weight vector may be increased as additional amplified samples are received.

According to aspects, the memory may have further instructions stored thereon which, when executed by the processor, cause the computer to transmit a control signal based on the prediction in a case where the prediction satisfies prediction criteria.

In aspects, the memory may have further instructions stored thereon which, when executed by the processor, cause the computer to determine if a subset of signals is associated with a flat channel, and in a case where the subset of signals is determined to be associated with a flat channel, discard the subset of signals.

According to aspects, the memory may have further instructions stored thereon which, when executed by the processor, cause the computer to, initialize a weight vector having a weight associated with the classifier, the weight corresponding to a probability that the classifier is reliable.

In aspects, additional signals are amplified by the biosignal acquisition device and transmitted to the computer.

According to aspects, the memory may have further instructions stored thereon which, when executed by the processor, cause the computer to generate a second feature matrix based on the additional amplified signals, identify the brain switch associated with each feature of the second feature matrix, predict the probability that a real-time classifier is associated with the brain switch of each feature, fit the real-time classifier based on the second feature matrix, and refit the classifier based on the fitting of the real-time classifier.

According to embodiments of the present disclosure, a biosignal acquisition system for identifying brain switches and transmitting context commands based on the brain switches is disclosed. The system may include a computer, a biosensor, and a biosignal acquisition device. The biosensor may be configured to be in contact with a scalp of an individual. The biosignal acquisition device may be operably coupled to the biosensor and configured to amplify signals received by the biosensor and communicate the amplified signal to the computer. The computer may include a processor and memory having instructions stored thereon which, when executed by the processor, cause the computer to receive the amplified signals from the biosignal acquisition device, determine if a subset of signals are associated with a channel having a deficient signal, in a case where the subset of signals are determined to be associated with a flat channel, discard the subset of samples, generate a feature matrix based on the remaining amplified signals, identify a brain switch associated with each feature in the feature matrix, predict the probability that a classifier is associated with the brain switch of each feature, and fit the classifier based on the feature matrix.

In aspects, the memory may have further instructions stored thereon, which when executed on the processor, cause the computer to determine the quality of the deficient biosensors relative to a set of non-deficient biosensors based on comparing the signals associated with the deficient biosensors and the signals associated with the non-deficient biosensors, the set of non-deficient biosensors comprising the biosensors that not identified as deficient.

According to embodiments of the present disclosure, a biosignal acquisition system for identifying context commands and controlling a computing device based on the identified context commands is disclosed. The system may include a computer, a biosensor and a biosignal acquisition device. The biosensor may be configured to be in contact with a scalp of an individual. The biosignal acquisition device may be operably coupled to the biosensor and configured to amplify signals received by the biosensor and communicate the amplified signal to the computer. The computer may include a processor and memory having instructions stored thereon which, when executed by the processor, cause the computer to receive the amplified signals from the biosignal acquisition device, generate a feature matrix based on the amplified signals, identify a brain switch associated with each feature in the feature matrix, predict the probability that a classifier is associated with the brain switch of each feature, and fit the classifier based on the feature matrix. A computing device may be in electrical communication with the computer, the computing device having a processor and memory having instructions stored thereon which, when executed by the processor, cause the computer to receive a control signal based on a context map stored in the computer and the prediction performed by the computer, and perform a function based on the received controls signal.

According to aspects, the memory of the computing device may further have instructions stored thereon which, when executed by the processor of the computing device, cause the computing device to transmit a control signal to the computer to swap the context map for a second context map.

According to embodiments of the present disclosure, a method of transmitting context commands based on sensed biosignals is disclosed. The method may include receiving a first set of signals from an electrode in close proximity to a scalp of an individual, generating a feature matrix based on the first set of signals, identifying a brain switch associated with each feature in the feature matrix, predicting the probability that a classifier is associated with the brain switch associated with each feature, and fitting the classifier based on the feature matrix.

In aspects, the method further includes initializing a weight vector having a weight associated with the classifier, the weight corresponding to a probability that the classifier is reliable.

According to aspects, the method includes receiving a second set of signals, generating a second feature matrix based on the second set of signals, identifying a brain switch associated with each feature of the second feature matrix, predicting the probability that a real-time classifier is associated with the brain switch of each feature of the second feature matrix, fitting the real-time classifier based on the second feature matrix, and refitting the classifier based on the fitting of the real-time classifier.

In aspects, the method includes transmitting a control signal based on the prediction in a case where the prediction satisfies prediction criteria.

According to aspects, the method includes determining if a subset of signals is associated with a flat channel, and in a case where the subset of signals is determined to be associated with a flat channel, discarding the subset of samples.

In aspects, the method includes initializing a weight vector having a weight associated with the classifier, the weight corresponding to a probability that the classifier is reliable.

In embodiments, a method of transmitting control signals based on sensing one or more brain switches is disclosed. The method may include receiving amplified signals from a biosignal acquisition device, generating a feature matrix based on the amplified signals, identifying a brain switch associated with each feature in the feature matrix, predicting the probability that a classifier is associated with the brain switch of each feature, fitting the classifier based on the feature matrix, transmitting a control signal based on a context map and the predicting, and performing a function based on the transmitted control signal.

### BRIEF DESCRIPTION OF THE DRAWINGS

The accompanying drawings, which are incorporated in and constitute a part of the specification, illustrate embodiments of the disclosure and, together with a general description of the disclosure given above, and the detailed description of the embodiment or embodiments given below, explain the principles of the disclosure.

FIG. 1 is a schematic view of a biosignal system including a biosignal acquisition headset, in accordance with the present disclosure;

FIG. 2 is a schematic block diagram of a computing device that may be employed according to various embodiments of the present disclosure;

FIG. 3A is perspective view of the biosignal acquisition headset of FIG. 1;

FIG. 3B is an exploded perspective view of the biosignal acquisition headset of FIG. 1;

FIG. 4 is flow diagram of a method for identifying and associating biosignals with certain thoughts in accordance with aspects of the present disclosure;

FIG. 5 is flow diagram of a method to identify what an individual 101 is focused on based on collected biosignals, in accordance with aspects of the present disclosure;

FIG. 6 is a flow diagram of a method for determining the signal quality of multiple biosignals, in accordance with aspects of the present disclosure;

FIGS. 7A and 7B area flow diagrams of a method for determining the signal quality of multiple biosignals in real-time, in accordance with aspects of the present disclosure;

FIG. 8 is a flow diagram of a method for training an individual 101 to differentiate between differing thoughts, in accordance with aspects of the present disclosure;

FIGS. 9A and 9B are a perspective views of the biosensor of the biosignal acquisition headset of FIG. 1;

FIG. 10 is a perspective view of the biosensor mold that of the biosignal acquisition headset of FIG. 1;

FIGS. 11A-11D, are views showing examples of identifying a stimulus to an individual 101 in respective processes;

FIG. 12 shows an example of training a classifier in respective processes;

FIG. 13 are views showing examples of combining multiple classifiers to produce a single probability in respective processes;

FIG. 14 is an example of determining the signal quality of multiple biosignals in respective processes; and

FIG. 15 is an example of determining a the signal quality of multiple biosignals in respective processes.

### DETAILED DESCRIPTION

The present disclosure is directed to systems and methods of collecting, analyzing, and associating biosignals with certain thoughts as such thoughts are recalled by the brain of an individual 101. These biosignals are associated with instructions which may be executed on a computing device upon recall of the thought by the individual 101.

As used herein, the terms “End User” and “individual” are used interchangeably to refer an individual 101 who is operating the systems and methods of the present disclosure; the term “thought” denotes any repeatable impulse or oscillation of biosignal activity; the terms bioelectrical signal or biosignal are used to refer to electrical signals which are discharged by a brain as neurons in the brain fire and which may be measured and graphed via electrodes in contact with the scalp of an individual 101 (e.g., an electroencephalogram (“EEG”). These biosignals along with any ancillary electrical artifacts (or abnormalities, e.g., electric activity of muscle (shown on an electromyogram (“EMG”)), eye movement (shown on an electro-oculogyric (“EOG”))) may be received or measured by electrodes in proximity to the scalp of the individual 101.

The present disclosure describes systems and methods for more accurately detecting neural commands associated with thoughts of an individual 101. Additionally, the present disclosure describes, in embodiments, systems to be used among multiple individual 101s which, over time, aggregate a plurality of biosignals and identify patterns across the identified biosignals. The detection of a thought in an individual 101 may trigger a plurality of actions based on what the individual 101 may be focused on. More particularly, the individual 101(s) may consciously invoke a biosignal which, when recognized by the biosignal acquisition headset, causes control signals to be transmitted to execute one or more instructions on a remote device (e.g., a computer, a mobile device, and the like).

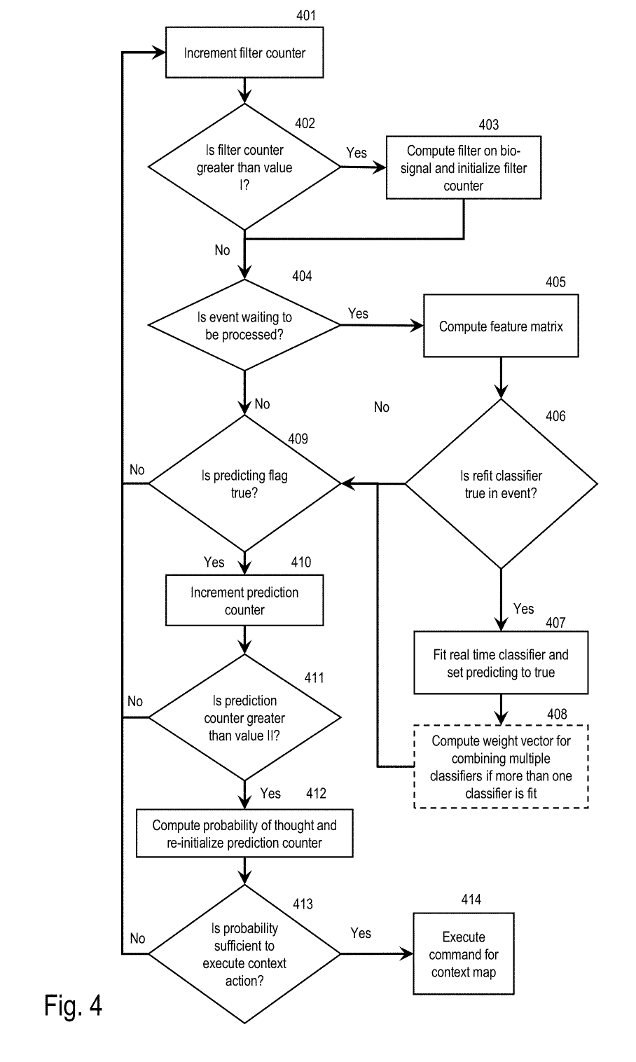

The following example, which is not intended to be limiting and is provided for illustrative purposes, describes use of a system to recognize, classify, and use biosignals in accordance with aspects of the present disclosure. An individual 101 may associate or link one focused thought (e.g., the thought moving a tongue in a particular manner, lifting the right toe, etc.), referred to herein as a “brain switch,” to one or more executable functions or commands. Such commands may include instructions which, when executed on a computing device, cause the computing device to perform one or more actions (e.g., open a web browser, send a message, etc.). In use, the individual 101 first invokes the thought of moving their tongue multiple times during a calibration phase while being monitored by a headset 300 of a biosignal acquisition system 100. Once calibrated the headset 300 then, in use, causes one or more functions to be triggered when the brain switch is recognized, thereby causing the instructions to be executed on the computing device. A more detailed description of calibration is provided in detail with respect to FIG. 4. The individual 101 does not need to physically move their tongue or lift their right toe to effectuate the brain switch, rather, they may effectuate the brain switch by thinking about the movement. The individual 101 may link the brain switch to tasks such as changing the episode on Nedlix®, composing an email in Gmail®, and scrolling down on the page when reading an article in Wall Street Journal Online®.

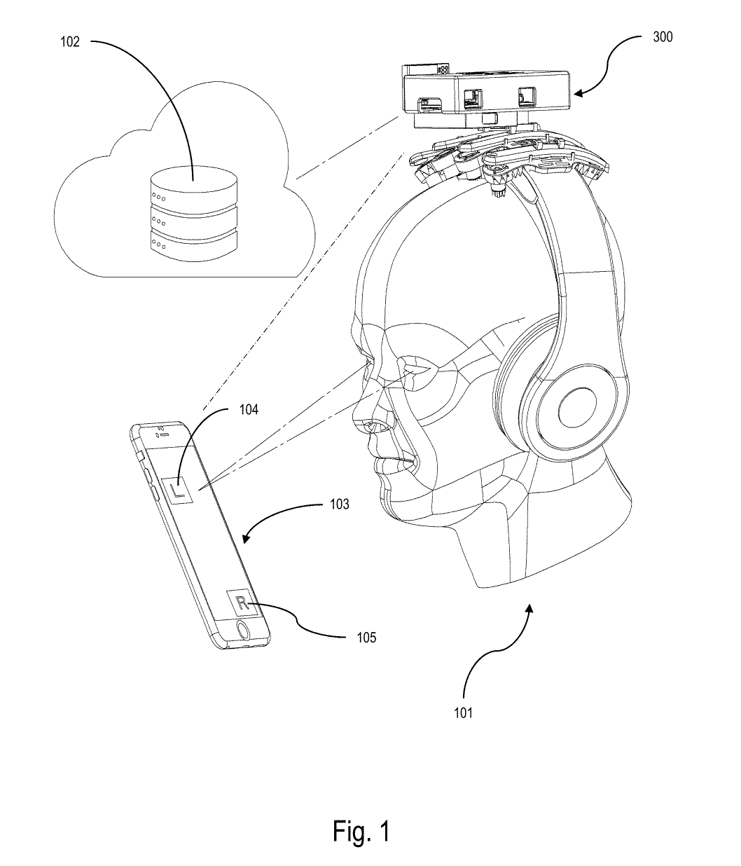

As illustrated in FIG. 1, a biosignal acquisition system 100 includes a headset 300, a database 102 in electrical communication with the headset 300, and a remote computing device or mobile device 103. The headset 300 is configured to be disposed on the scalp of an individual 101. While in operation, the headset 300 is configured to recognize and analyze biosignals for brain switches from the individual 101. In embodiments, the database 102 may be stored in memory 204 of the headset 300, or in the memory 204 of the mobile device 103.

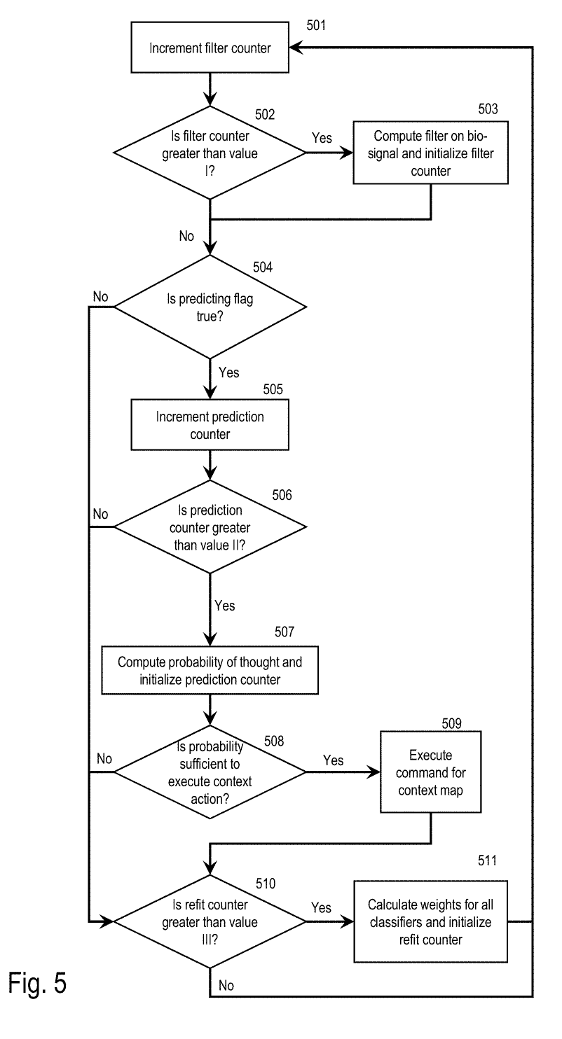

An individual 101 affixes the headset 300 to a top portion of the head of the individual 101. The headset may start process 700, to determine the quality of each biosensor and request and adjustment of the headset 300 on the scalp of the individual 101. The headset 300 may connect to a database 102 to save classifiers produced by process 400 and context maps used in process 600. The headset may load classifiers from memory 204 of any of the computer 309, database 102, or any other connected computing device and/or memory storage device produced by process 400 or context maps used in process 600. For purposes of clarity, reference will be made to the memory 204 of the computer 309, however, it will be understood that the database 102 or any other suitable computing device may be used to execute the methods described herein. Process 400 may be started automatically if there is less than a predetermined number of classifiers loaded from memory 204 of the computer 309. If greater than a predetermined number of classifiers are retrieved from memory 204 on computer 309 or a database 102, then process 500 may be started which uses an algorithm (e.g., transfer learning, see process 500, FIG. 5) to combine predictions from multiple classifiers to compute a probability of a thought without the use a training or calibration process. Process 400 and process 500 may produce probabilities for an individual 101 thinking a thought. A headset 300 may be producing the probability of multiple thoughts at any given time. Each thought may be assigned to a particular action that may be executed if the probability exceeds a particular predetermined set of criteria. Process 600 may be used to derive what an individual 101 may be focused on using the context maps loaded from memory 204 of the computer 309 or a database 102 and change the action assigned to a thought.

The database 102 may store one or more classifiers in memory 204 of the database 102. As used herein, the term “classifier” denotes a function or executable instructions that, when executed, predicts the probability of a learned biosignals being associated with a brain switch. The headset 300 may require authentication to retrieve the classifiers and metadata from database 102, so as to secure the classifiers which are generated for an individual 101 or group of individuals 101. In embodiments, database 102 may be located remote relative to the headset 300, while in either wired or wireless electrical communication with the headset 300. In embodiments, the individual 101 may control one or more biosignal acquisition systems 100 by invoking the learned biosignals associated with the biosignal acquisition system 100. More particularly, the individual 101 may associate certain brain switches, and by extension biosignals, with one or more commands to be used in a environment (e.g., a set of commands to control a computing device at work, at home, when exercising, etc.).

With continued reference to FIG. 1, a display of the mobile device 103 may be used during calibration to verify a brain switch. For example, as the individual 101 invokes the thought of lifting their right toe, the biosignal acquisition system 100, may recognize the biosignals received by the headset 300. For every biosignal captured which indicates that a particular brains switch is being invoked, the headset 300 may cause a left block 104 on the display of the mobile device 103 to move upward on the screen. Conversely, the learned brain switch associated with a right block 105 may cause the right block 105 to move less or not at all when the probability of identified brain switch being associated with the right block 105 is low.

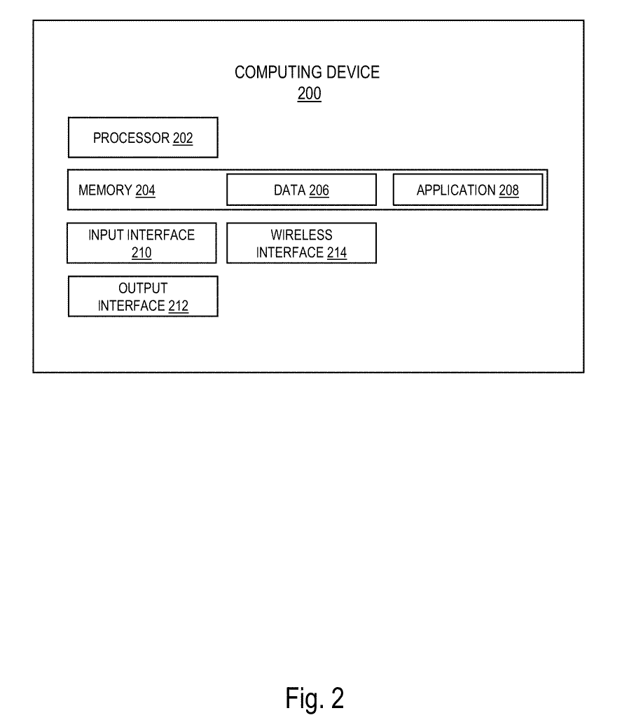

FIG. 2 is a schematic block diagram of a computing device 200 that may be employed according to various embodiments of the present disclosure. Though not explicitly shown in corresponding figures of the present application, the computing device 200, or one or more components thereof, may represent one or more components (e.g., a controller disposed within the headset 300, the mobile device 103, the database 102, etc.) of the biosignal acquisition system 100. The computing device 200 may include one or more processors 202, memories 204, input interfaces 210, output interfaces 212, wireless interfaces 214, or any desired subset of components thereof.

The memory 204 includes non-transitory computer-readable storage media for storing data and/or software which includes instructions that may be executed by the one or more processors 202. When executed, the instructions may cause the processor 202 to control operation of the computing device 200, e.g., reception and transmission of sensor signals and/or control signals during operation of the computing device 200. In embodiments, the memory 204 includes non-transitory computer readable storage media for storing data and/or software which includes instructions that may be executed by the one or more processors 202. The memory 204 may include one or more solid-state storage devices such as flash memory chips. Additionally, or alternatively, the memory 204 may include one or more mass storage devices in communication with the processor 202 through a mass storage controller and a communications bus (not shown). Although the description of computer readable media described in the present disclosure refers to a solid-state storage device, it will be appreciated by one of ordinary skill that computer-readable media may include any available media that can be accessed by a processor 202. More particularly, computer readable storage media may include, without limitation, non-transitory, volatile, non-volatile, removable, non-removable media, and the like, implemented in any method of technology for storage of information such as computer readable instructions, data structures, program modules, or other suitable data access and management systems. Examples of computer-readable storage media include, RAM, ROM, EPROM, EEPROM, flash memory, or other known solid state memory technology, CD-ROM, DVD, Blu-Ray, or other such optical storage, magnetic cassettes, magnetic tape, magnetic disk storage or other magnetic storage devices, or any other medium which may be used to store information and which can be accessed by the computing device 200.

In embodiments, the memory 204 stores data 206 and/or one or more applications 208. The applications 208 may include instructions which are executed on the one or more processors 202 of the computing device 200. The applications 208 may include instructions which cause an input interface 210 and/or an output interface 212 to receive and transmit sensor signals, respectively, to and from associated devices. When received, the signals may be stored in the at least one memory 204 of the computing device 200. Additionally, or alternatively, the computing device 200 may transmit the signals for analysis and/or display via the output interface 212. For example, the output interface 212 may transmit the sensor signals to the display of the mobile device 103. The memory 204 may further transmit and/or receive data via wireless interface 214 via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 802.15.4-2003 standard for wireless personal area networks (WPANs)). Although depicted as a separate component, the wireless interface 214 may be integrated into the input interface 210 and/or the output interface 212.

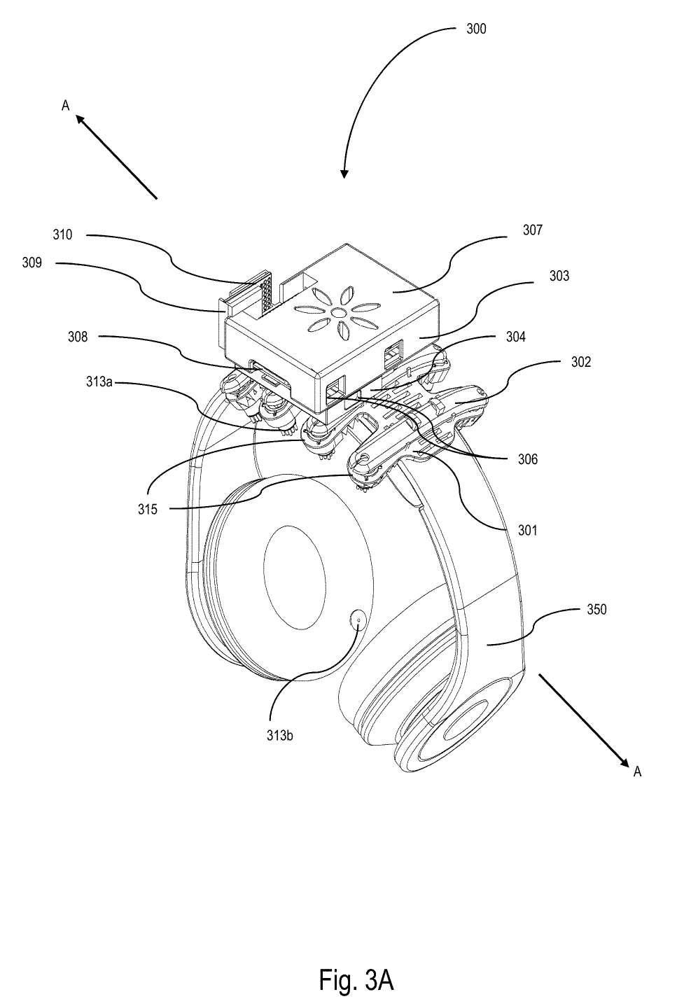

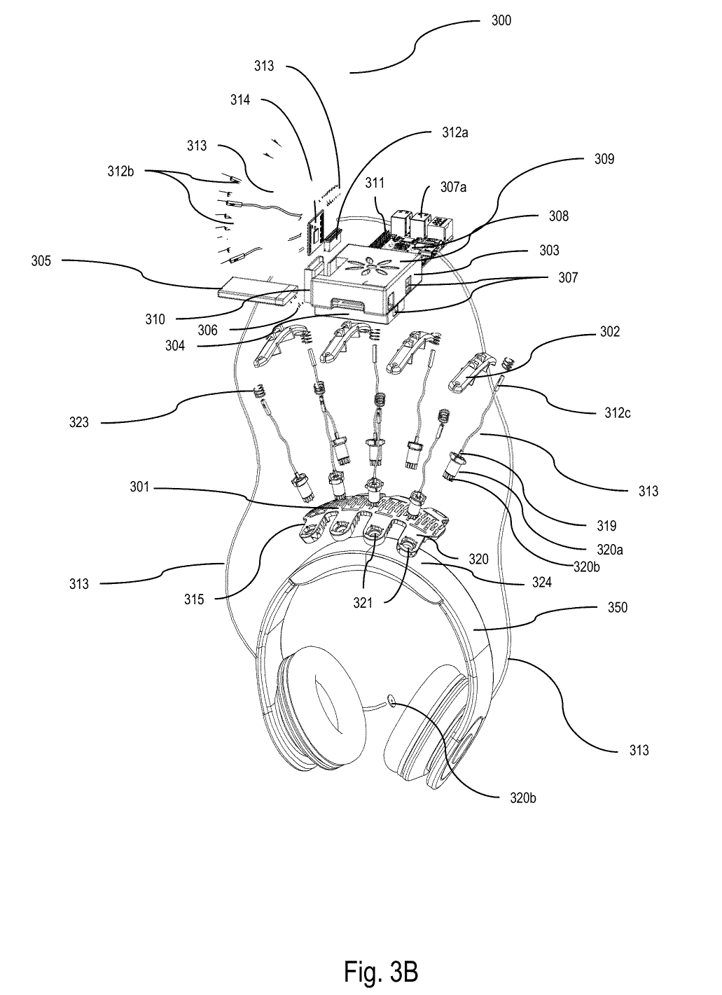

Referring now to FIGS. 3A and 3B, illustrated is a headset 300 for sensing biosignals. For illustrative purposes, a flexible membrane 301 is affixed to a headwear 350, however, in embodiments, the flexible membrane 301 may be affixed to any suitable structure capable of maintaining the flexible membrane in fixed relation to the scalp of the individual 101 (e.g., a hat, helmet, etc.).

The flexible membrane 301 may be made of any suitable semi-rigid or malleable material capable of contouring to, and being disposed on, a mounting surface the headwear 350 (e.g., silicone, hard rubber, etc.). In embodiments, the flexible membrane 301 may be fixed to a hat (not explicitly shown), or other suitable headwear known in the art as capable of maintaining the position of the flexible membrane 301 relative to the head of the individual 101. As illustrated in FIG. 3A, the flexible membrane 301 extends along an axis A-A which extends from the right portion of the head of the individual 101 to the left portion. While it is contemplated that the orientation of the flexible membrane 301 relative to the individual 101 may be in any direction, for purposes of clarity, continued reference will be made to the left-to-right (transverse) orientation of the flexible membrane 301 relative to the individual 101. In embodiments, the flexible membrane 301 may be configured to remain in place on the head of the individual 101 without the support of headwear 350 or, alternatively, when integrated into headwear 350.

The flexible membrane 301 includes a plurality of arms 315 extending from the center of the flexible membrane 301 outward relative to the axis A-A. The arms 315 are configured to couple to cross membranes 302 in any suitable manner such as, without limitation, with screws, fasteners, via friction fits, snap fits, and the like. The flexible membrane 301 support an electronics housing 303 thereon and maintains the position of the electronics housing 303 relative to the individual 101. In embodiments, the flexible membrane 301 may be located anywhere on an individual 101 or animal body where originating biosignals from the brain may be measured with an electrode or an array of electrodes. The biosensor 320s are configured to couple to arms 315 in any suitable manner such as, without limitation, with screws, fasteners, via friction fits, snap fits, and the like. For example, the flexible membrane 301 may be affixed to an arm of an individual 101 to capture biosignals produced by the brain which cause the hand of the individual 101 to clinch into a fist.

Cross membranes 302 may be formed of any suitable semi-rigid or malleable material capable of contouring, and/or coupling, to the flexible membrane 301 (e.g., silicone, rubber, plastic, etc.). The cross membranes 302 may, in embodiments, be positioned asymmetrically along either side of the axis A-A to better contour the scalp of an individual 101. The cross membrane 302 may be interchangeable to conform to varying head shapes of individual 101s 101. In embodiments, the cross membranes 302 may be rotatably coupled to the flexible membrane 301, or may be divided into half-portions or arms which extend forward or backward. The arms may be rotatably coupled to the flexible membrane 301 for selective positioning relative to the scalp of the individual 101.

The electronics housing 303 is coupled to the flexible membrane 301. As illustrated in FIGS. 3A and 3B, the electronics housing 303 includes a lower housing portion 304, an intermediate housing portion 308, and a side housing 310. The electronics housing 303 may be affixed to the flexible membrane 301 or cross membrane 302 in any suitable manner (e.g., by fastening, gluing, attaching via a friction or snap fit connection, etc.).

The lower housing portion 304 may include a power supply 305 and a mounting surface 324 configured to connect to the flexible membrane 301 and the cross membrane 302. The lower housing portion 304 may contain conduits 307 which enable one or more wires (e.g., wires 306, 313 to extend through the lower housing portion 304 to the electronics disposed therein. The power supply 305 stores electrical energy and regulates delivery of the electrical energy to the headset 300 via wire 306, and more particularly to the components of headset 300, which include, without limitation a computer 309 and a biosignal acquisition device 314. The power supply 305 may contain a plurality of batteries (not explicitly shown) and may be recharged through a power interface. As illustrated, a plurality of conduits 307 are disposed about the lower housing portion 304 and intermediate housing portion 308. In embodiments, a plurality of conduits 307 may be disposed along the lower housing portion 304, the intermediate housing portion 308, and the side housing 310. The conduits 307 allows connectors 312 and/or wires 313 to extend through the housing to connect to the biosignal acquisition device 314.

The intermediate housing portion 308 may include a computer 309 disposed therein. The intermediate housing portion 308 may contain a plurality of conduits 307 configured to accept one or more connectors 307a, for example, a plurality of USB-A plugs, USB-C plugs, etc. The intermediate housing portion 308 may further be configured to receive one or more plugs disposed along a power cable when the power supply 305 is being recharged, when updating firmware, transferring context maps, classifiers or other data to the memory of the computer 309, etc.

The computer 309 may be an embedded computing system similar in many respects to the computing device 200 (FIG. 2). A headset 300 may be comprised of a plurality of computers 309 that may execution instructions simultaneously and collaboratively. In embodiments, the computer 309 may be configured to run multiple processes at the same time (process in parallel) to reduce the amount of hardware housed within the electronics housing 303. The computer 309 may be powered by the power supply 305 via wire 306. The computer 309 may have a plurality of interfaces 311 that may be connected to a plurality of biosignal acquisition devices 314 via connectors 312 and/or wires 313. Additionally, or alternatively, the computer 309 may wirelessly communicate with a plurality of biosignal acquisition devices 314. The computer 309 may contain integrated memory and/or removable memory 204 (FIG. 2), a processor 202, graphics processor (not explicitly shown), and a general-purpose input/output (GPIO) port (not explicitly shown). The computer 309, and more particularly the processor 202 and/or the graphics processor, may have multiple CPU or GPU cores, respectively, such that process 400 for classifier learning for predicting thoughts, process 500 for combining multiple classifiers for predicting thoughts, and process 700 for evaluating the quality of biosensor connection may be executed simultaneously.

The side housing 310 may be further comprised of a material such as graphene infused plastic, any metallic metal that may be conductive in its physical properties, metal shielding layer to reduce electrical noise interference, such as electromagnetic interference, from affecting the components included therein. In embodiments, the side housing 310 may have one or more conduits 307 disposed thereon to permit passage of connectors 312 and/or wires 313, therethrough.

The connectors 312 may be any rigid, or semi-rigid material that contain one or more wires which transmit digital voltages as data signals. For illustrative purposes, the device connector 312 may transmit control and data line connections to enable communication between the computer 309 and the biosignal acquisition device 314. The connector 312 may connect a plurality of wires 313 together. As illustrated in FIG. 3B, connector 312b may be physically connected connector 312c which allows a plurality of electrical signals to propagate freely through via any rigid, or semi-rigid electrically conductive material or plating. Connector 312a may be used to connect a biosensor 320 to an interface 311. Connector 312 may be an electromechanical solder connection that may allow wire 313 to directly touch an interface 311.

The wire 313 may be replaced with a wireless interface 214 (FIG. 2) which transmits wireless signals such as Bluetooth or WiFi (802.1) signals. The wireless signals may include sensor (data) and/or control signals and/or metadata algorithmically derived from the sensor (data) transmitted from the biosignal acquisition device 314 to the computer 309 as illustrated in FIG. 3A via the absence of wires 313 between biosignal acquisition device 314 and computer 309. The wire 313 may be made of a plurality of different wire configurations such as a strand of wires bundled together wrap in an insulating material such that the wire 313 may be flexible and absorb minute vibrations that may cause noise if a solid metal core wire was used.

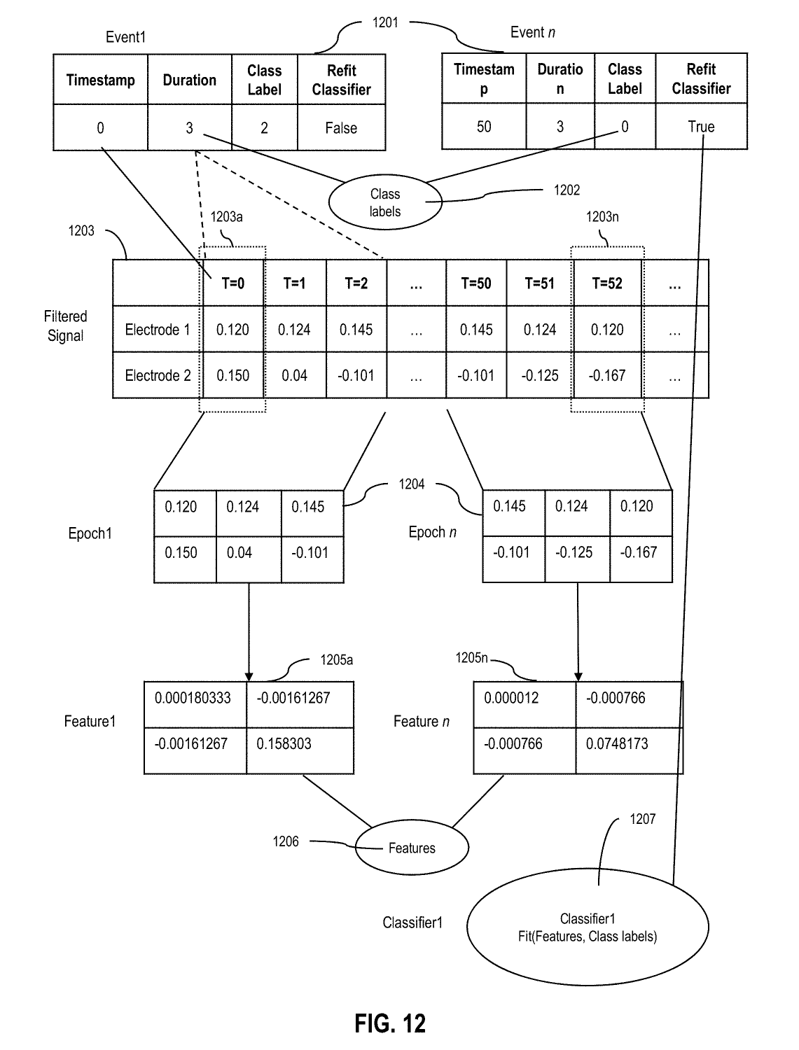

The biosignal acquisition device 314 may be an analog to digital voltage converter that computes a digital representation of a physical measurement in a periodic cycle, generally referred to as a sample. A “sample”, is a collection of biosensor values sampled at the same point in time, an example for illustrative purposes may be found is sample 1203a on FIG. 12. The biosignal acquisition device 314 may be connected to the computer 309 and/or biosensors 320 via wireless connections (not explicitly shown). As illustrated in FIG. 3B, biosignal acquisition device 314 is connected to the computer 309 via the interface 311. The interface 311 is operably coupled to wires 313 and may be connected via a connector 312a. The biosignal acquisition device 314 may be connected to a biosensor 320a and/or biosensor 320a via a wire 313 such that there is no connector 312 used. Additionally, the wires 313 may use male connector 321b to operably connect to female connector 312c as depicted in FIG. 3B. A plurality of biosensors 320 may be connected to biosignal acquisition device 314 via a wireless connection (not explicitly shown), a plurality of wires 313 or a plurality of connectors 312 and wires 313. The biosignal acquisition device 314 may be contained within side housing 310.

The biosensor 320 may be connected to bio-signal acquisition biosignal acquisition device 314 via a wireless connection, through a wire 313, or through a plurality of connectors 312 and wires 313, thereby permitting analog voltages to be measured or control and/or digitized biosensor data signals to be transmitted therebetween. For illustrative purposes only, depicted in FIG. 3B, the biosensor 320a is electrically connected to connector 319 through a screw (not explicitly shown), the connector 319 is electrically coupled with wire 313 to a female connector 312c which passes through conduit 307 and electromechanically connects to male connector 312b, which is then electromechanically connected to bio signal acquisition device 314, thereby permitting control and/or data signals to be transmitted therebetween. The wire 313 may include a plurality of wires, not explicitly shown, enabling electrical communication between biosensor 320a and biosignal acquisition device 314 and a biosensor 320 that is active. For a detailed description of an active electrode, reference may be made to U.S. patent Ser. No. 13/619,524 entitled “Active Dry Sensor Module for Measurement of Bioelectricity,” the contents of which are hereby incorporated by reference in their entirety. Active electrode hardware may be housed internally in in biosensor 320a.

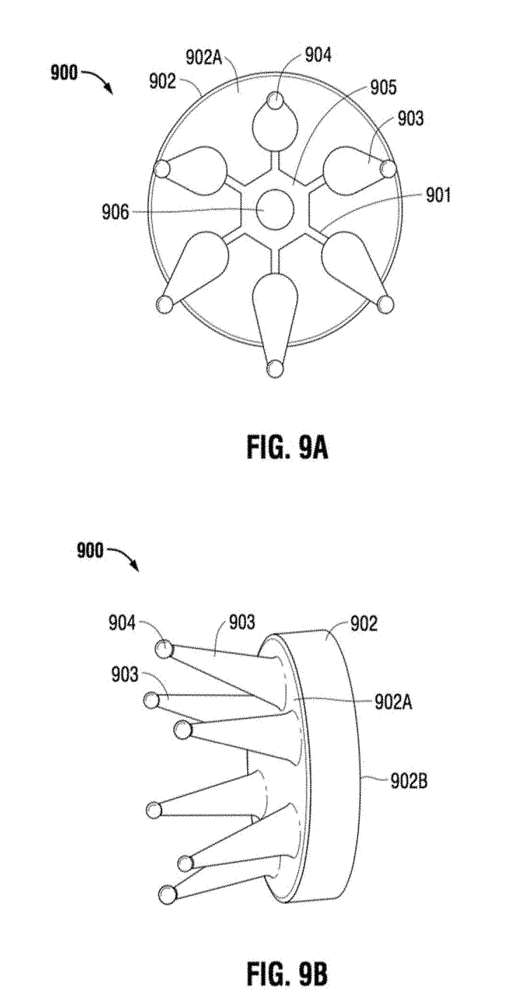

The biosensor 320 may be any suitable electrode which is configured to detect biosignals on any animal. The biosensor 320a may be a pronged electrode having a length configured to extend through the hair of the individual 101. The length of the prongs 320b of the biosensor 320a may be any length, (e.g., from 1 mm to 50 mm) determined to be long enough to place the biosensor 320a in contact with the scalp of the individual 101 (FIG. 1). As illustrated, the biosensor 320a (FIGS. 9A and 9B) may, in embodiments, rest flat against the head of the individual 101 when the individual 101 has little to no hair which would impede contact between the biosensor 320a and the scalp of the individual 101. The biosensor 320a may be made of a plurality of different materials such as chopped carbon fiber randomly interspersed silicone or graphene mixed with plastic to transfer ion currents to connector 319. The portion of biosensor 320a that configured to contact the scalp of the individual 101 may be made of any bio-compatible material such as, without limitation, silver (Ag) or gold (Au) or silver/silver-chloride (Ag/AgCl), which improves the ion to electron conversion needed to digitize biosignals on biosignal acquisition device 314. The biosensor 320 may be any suitable sensor configured to detect biosignals such as, without limitation, electrically conductive sensors, or optically emitting and detecting sensors. The biosignal acquisition device 314 may be interchangeable based on the type of biosensor 320 selected for a particular application. For a detailed description of optically emitting and detecting, reference may be made to U.S. Pat. No. 6,291,824 entitled “Apparatus and Method for High-Bandwidth Optical Tomography,” the contents of which are hereby incorporated by reference in their entirety.

Spring 323 may provide outward force to the flexible membrane 301 and the cross membrane 302. The spring 323 may not be required for all applications and may be used to improve contact between the biosensor 320a and the scalp of the individual 101.

Mounting surface 324, may be part of headwear 350, in embodiments, may be the scalp of the individual 101, where the flexible membrane 301 is affixed to the scalp of the individual 101. As shown in FIG. 3B, mounting surface 324 is shown as a pair of headphones (referred to herein as “headwear 350”), however, such illustration should not be viewed as limiting the present disclosure. It is contemplated that, in embodiments, headwear 350 may be a hat, or other such head-mountable equipment known in the art as being capable of supporting the flexible membrane 301 relative to the scalp of an individual 101 (such mounting not explicitly shown). For example, the mounting surface 324 may be integrated into headphones (FIG. 1), hardhats, virtual reality headsets, augmented reality headsets, and the like.

Referring now to FIG. 4, illustrated is a method for detecting and associating or identifying specific brain switches (e.g., specific thoughts) of an individual 101 with biosignals (e.g., analyzed brain waves or brain activity associated with a particular brain switch). The identified biosignals may subsequently be used to control one or more systems (e.g., the mobile device 103, headset 300, or any other remote device configured to receive controls based on the identified brain switches) by receiving and executing the control signals based on the generation of the control signals.

For example, the individual 101 may think or generate a certain thought (e.g., lifting a big toe) which causes particular brain waves to be generated by the individual 101. Analog signals associated with brain waves or brain activity of the individual are captured by the biosignal acquisition device 314 (FIGS. 3A and 3B) and converted from analog signals to digital signals. The digital signals are transmitted to the computer 309 as biosignals which then are matched with one or more predefined control signals. Once matched, the computer 309 may cause the computer 309, or any other computing device associated with the biosignal acquisition device 314 (e.g., mobile device 103) to execute instructions.

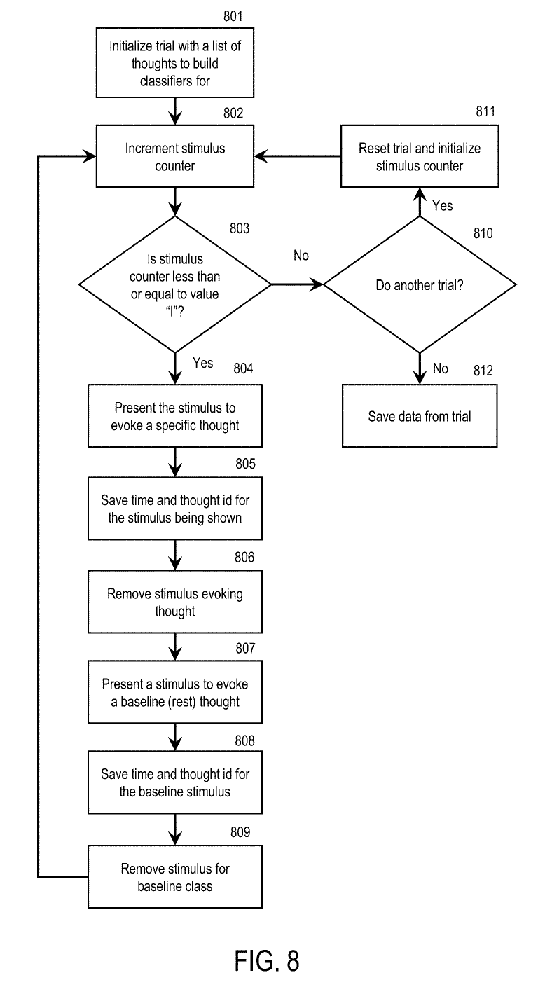

Described now is a process of sensing and learning (or associating) brain switches with control signals. The process is generally referred to as classifier training (herein “process 400”). While process 400 may be executed on any suitable computing device (see computing device 200; FIG. 2), for purposes of clarity, the processes described herein will be referred to as being implemented on the computer 309 of the headset 300 (FIG. 1). The classifiers generated (or “fit”) by process 400 may be stored in and/or associated with a particular individual and/or device. The classifiers produced by process 400 may also be identified and refit by a different process in accordance with the present disclosure (e.g., process 800) (see FIG. 8).

Initially, a new discrete sample sensed by the biosensor 320a is received as an analog signal by the biosignal acquisition device 314 and converted to a digital representation of the signal (e.g., 4.5 uV), the sample potentially corresponding to a brain switch (e.g., a thought) occurring at a particular time (block 401; see table 1203 illustrating a sample ring buffer having values taken from time 0 to time 52). The sample may include measurements collected from one or more biosensors 320a. Once received by the biosignal acquisition device 314, the samples are transmitted to the computer 309 to be indexed and stored in the memory 204 of the computer 309. In embodiments, the samples are transmitted to a remote computing device (e.g., database 102) for storage and/or analysis. The biosignal acquisition device 314 or the computer 309 may also associate each sample with a system time stamp.

Each sample is then placed into a ring buffer (e.g., a two-dimensional array of computer memory configured to store a predetermined amount of samples) in the memory 204 of the computer 309. If the ring buffer (FIG. 12, table 1203) is filled (e.g., there is no more room in memory for additional samples), the oldest sample is discarded from the memory 204 and the newest sample is placed into the zeroth index position (e.g., a first in, first out scheme is used to maintain the most recent samples in the memory 204), though it is contemplated that the memory 204 may be expanded to accommodate additional samples.

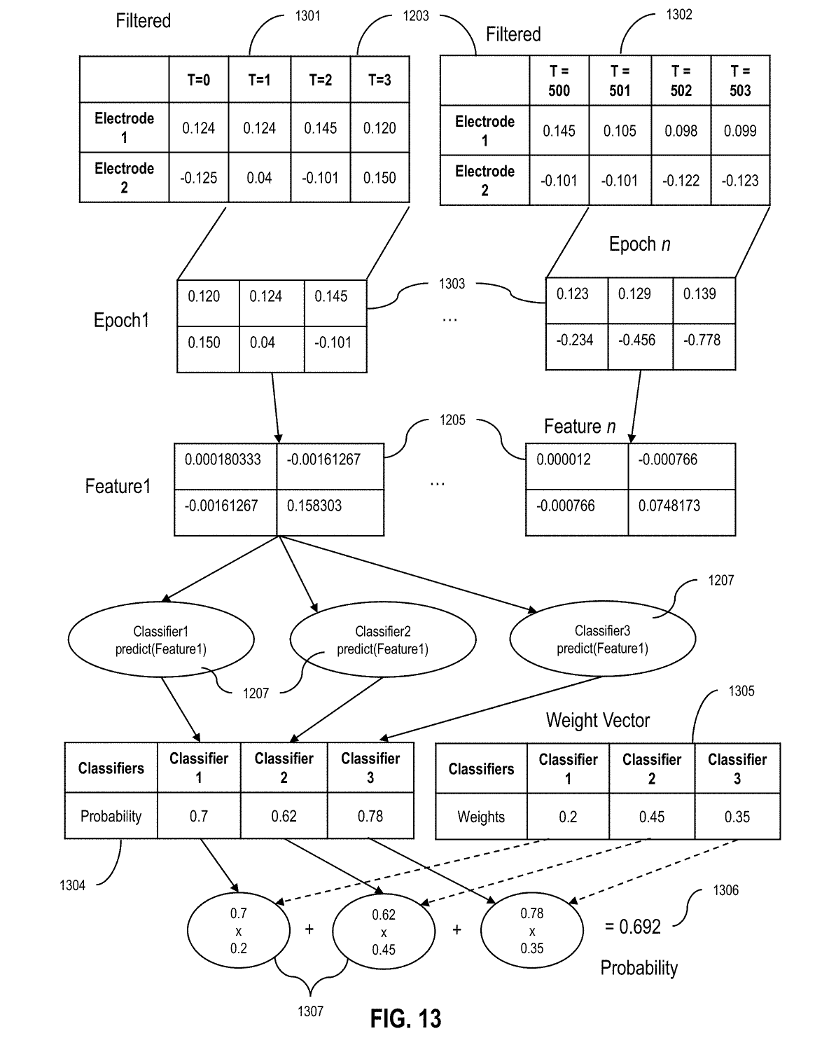

A prediction counter and filter counter are also initialized, the prediction and filter counters including indicators that a prediction is being made and that a number of samples have been filtered, respectively. A weight vector is also initialized, the weight vector representing corresponding classifier values that sum to 1.0 (or 100%, see table 1305, FIG. 13). Each classifier value stored in the weight vector corresponds to a weight indicating the level of influence which execution of a predict function with the information associated with the classifier should have in determining whether a series of samples indicates the presence of a brain switch. The active class label is associated with a predetermined brain switch (or thought type), for example, a first class label may be associated with the thought of “resting”, and a second class label may be associated with the thought of “closing the right hand of an individual.” The set of class labels available when process 400 is initialized are the class labels which will be fit throughout execution of process 400.

As used herein, the term “classifier” will be understood to refer to an algorithm that takes as input a set of features and class labels and learns and/or identifies a pattern between the sets of features. The classification algorithms employed may be any known classification algorithm in the field of brain computer interfaces. For example, to “fit” or “refit” a classifier, the geometric mean of aggregated features (e.g., the mean of the features from feature “1” to feature n, see FIG. 12, tables 1205a-1205n) associated with each class label may be computed and considered to be the center of a respective class within a Riemannian manifold. Each geometric mean is considered to be a measurable point in the Riemannian manifold. When a new sample forms a new epoch (a predetermined number of contiguous biosensor samples) a probability may then be determined by comparing the distance from the newly estimated covariance matrix to the previously computed center of each class. For a detailed description of a classifier, and system and methods of fitting classifiers, reference may be made to U.S. Patent No. 2012/0071780 entitled “METHOD AND A SYSTEM FOR CLASSIFYING NEURAL SIGNALS, AND A METHOD OF SELECTING ELECTRODES FOR DIRECT NEURAL CONTROL,” the contents of which are hereby incorporated by reference in their entirety.

The weight vector (see table 1305, FIG. 13) includes probability percentage values (or weights) associated with the reliability of a particular classifier when predicting that a brain switch associated with a corresponding class label is present among a plurality of samples (e.g., an epoch). The weight vector is initialized or calculated when the training (e.g., an instance of the execution of process 400 and/or 500) produces additional classifiers, or information to refit existing classifiers. In embodiments, the weight vector may be recalculated based on the passage of a predetermined amount of time. Additionally, the weight vector is recalculated when additional classifiers are identified (e.g., after a classifier is computed (or fit), refit, and/or received from a remote computing device) The weight vector includes values for each classifier present at the start of process 400, as well as a classifier for real-time classification (process 500). If additional classifiers are produced, (e.g., classifiers are subsequently downloaded from the database 102), the additional probability percentage associated with the classifier is added to the weight vector, and the weight vector is recalculated (see block 408). By default, if no classifiers are identified (e.g., only a real-time classifier is present), a weight vector having a single value (e.g., 1.0) is initialized. Process 400 is also initialized with class labels, including one rest class label associated with a brain switch generated during rest thought and one active class label associated with a prompted brain switch generated when a thought prompt is displayed. Process 400 is initialized including one active class label whose probability is later calculated (see block 412).

Initially, when a sample is received by the computer 309 from the biosignal acquisition device 314, the filter counter is incremented (block 401). The sample is added to the ring buffer of size value “I”. If it is determined that the filter counter was incremented to a value greater than a predetermined number of samples (block 402, e.g., on successive iteration of blocks of process 400), the samples are filtered (block 403) and the filter counter reinitialized as 0. The filtered samples are subsequently added to a filtered signal ring buffer.

When filtering (block 403), extraneous information or noise from each sample is removed from the sample (e.g., signals outside a predetermined frequency range are removed by passing the signals through a band-pass filter) and stored into a filtered signal ring buffer. The predetermined frequency range and/or threshold range may be stored in memory 204 of the computer 309 to filter the samples in the ring buffer as the ring buffer fills (block 401) instead of filtering the entire signal ring buffer (block 403), though filtering may be performed on the signal ring buffer if desired. As a result, multiple processes 400 may be executed simultaneously to pull from the same filtered signal ring buffer, thereby reducing redundant computations.

The memory 204 of the computer 309 receives and stores one or more events or discrete sample subsets (e.g., sample 1203a, FIG. 12) from the biosignal acquisition device 314 (block 404). Each event is associated with a class label, duration, and time stamp. The class label for each event may be determined to be one of the initial class labels initialized with process 400. When an event associated with a class label is matched with an available class label, the epoch (e.g., a predetermined number of contiguous samples stored in the filtered signal ring buffer) including the sample is identified as a feature matrix associated with the class label (block 405).

As noted above, an epoch (table 1204, FIG. 12) is formed by collecting a predetermined number of contiguous samples from the filtered signal ring buffer. The epoch may then be further transformed into a feature according to a predetermined set of mathematical conversions determined by the class label. For example, as shown in FIG. 12, where a covariance of the values stored in epoch table 1204 is estimated, a table 1205 is computed with the covariance between each electrode, the resulting table 1205 is commonly referred to as a covariance matric. For a detailed description of a covariance matrix, reference may be made to U.S. Patent Application Publication No. 2007/0133878 entitled “METHOD FOR CONSTRUCTING COVARIANCE MATRICES FROM DATA FEATURES,” the contents of which are hereby incorporated by reference in their entirety.

The epoch and feature are stored in the memory 204 of the computer 309 for use when fitting real-time classifiers. (See block 407.) The epochs and features may be stored to the database 102 to avoid costly computational time when process 400 and/or 500 are executed to fit a classifier on the database 102. The features generated from one or more executions of process 400 may be also be aggregated together for subsequent recall and/or analysis. The process of “fitting” or “building” a classifier refers to learning or matching what the features associated with a specific thought type have in common (e.g., what electrical activity patterns form across multiple aggregated samples) and how these features differ from the features of a different thought type. A “fit” or “built” classifier may then take as input a feature and produce a probability that the feature is one of the two thought types that the classifier was fit or built with.

For example, the classifiers may be fit from the aggregated features across multiple training sessions (see FIG. 8) for an individual 101, multiple individuals 101 engaging with the headset 300, and/or multiple individuals 101 using headsets 300 in remote locations. The database 102 may, in embodiments, store only aggregated features across multiple training sessions to build global classifiers (e.g., classifiers which are fit based on multiple features from one or more individuals 101 engaging one or more headsets 300 which are in communication with the database 102). The differences and/or similarities may be identified and associated with the classifier as identified patterns. Once associated, a predict function may be called, the predict function comparing known patterns to a new or fresh feature (e.g., a new plurality of analyzed samples). Depending on the similarity of the new feature to the pattern associated with the classifier, a prediction is made (e.g., a value is output which indicates the percent likely that the feature corresponds to the feature of the earlier identified pattern).

Events may have a flag indicating that a real-time classifier has or has not been refit, the flag set at block 405. For example, a class label with a symbolic identifier of 0 may represent a rest state, where the individual 101 is presented with a visual or auditory stimulus to create a baseline mental state that encompasses a certain rest or control activity such as, without limitation, reading, determining the solution to a math problem, watching a video, and the like. A class label with a symbolic identifier of 1 may be associated with a specific thought or action, such as hitting the breaks in a car.

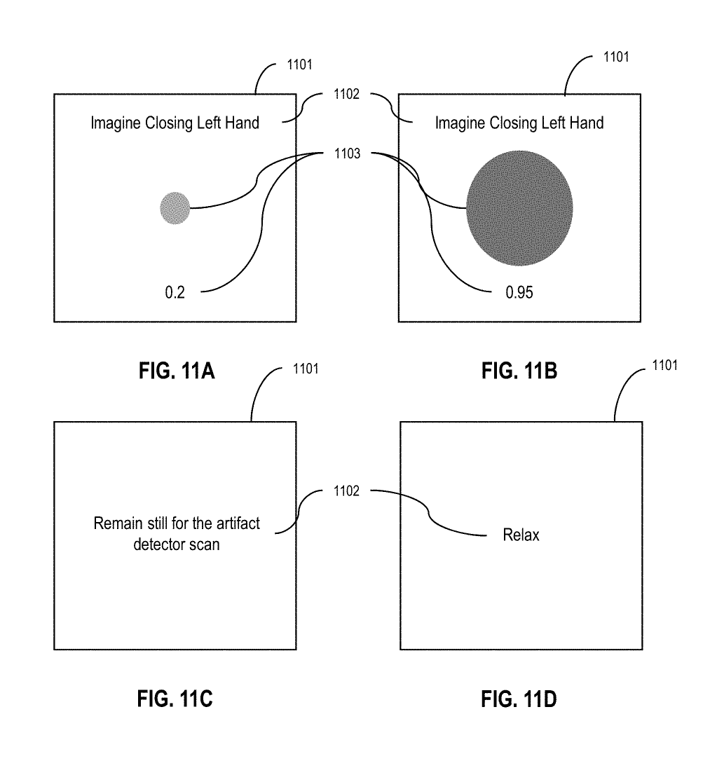

In embodiments, a predetermined amount of samples may be discarded when displaying or otherwise outputting visual stimuli. For example, during training a prompt associated with a class label having the identifier “1” may be displayed (FIGS. 11A and 11B) to cause the individual 101 to invoke the thought of closing a left hand. Since the individual 101 may anticipate a prompt associated with a class label “0” (e.g., a rest stimulus) (FIG. 11C and FIG. 11D), and begin to think about relaxing, samples and/or epochs collected within a predetermined amount of samples may be discarded to prevent false association of samples with an unassociated class label.

For example, if the stimulus shown to the individual 101 is not time dependent (e.g., an audible and/or visual instruction to imagine moving an arm) a brief period after the stimulus is shown to the individual may be observed to avoid extracting a feature from an epoch that contains samples that associated with the automatic response of the individual 101 to seeing an instruction flash in front of them. Additionally, the brief period may be observed (e.g., samples may be ignored or given less weight) to prevent leakage or contamination of desired brain switch samples with the previously invoked brain switch samples.

For example, the training application will show on a device 103 a stimulus for five seconds to an individual 101 to generate a specific thought associated with a specific class label. After five seconds have passed, the stimulus is removed and the current time, thought type, and duration will be formed into an event. The duration of the event may be less than the total time of actual stimulus presentation, e.g., duration of event is three seconds while stimulus presentation was five seconds. The samples associated with the unused portion (e.g., the first two seconds) may be discarded during classifier fitting.

To further improve the classifier fit in block 407, a method commonly referred to as bagging may be employed, where additional events are captured within a predetermined range of time after a stimulus is first shown (e.g., from seconds three to five of a five second interval, fifteen events may be identified, ten of which occur at random start times between seconds three and five). For a detailed description of bagging, reference may be made to U.S. Patent Application Publication No. US 2005/0071301 entitled “LEARNING SYSTEM AND LEARNING METHOD,” the contents of which are hereby incorporated by reference in their entirety. For illustrative purposes only, a stimulus is shown for five seconds to an individual 101, if bagging is utilized, a plurality of events may start to be created one second before the stimulus is removed with a duration of 3 seconds, e.g., 10 events with all the same duration and thought types, have random timestamps between the 4^{th} and 5^{th} seconds of the stimulus presentation, thereby producing 10 events instead of only one which may improve the classifier built by block 407.

If the refit flag (see example Table 1201 in FIG. 12) is determined to be true (block 406), the classifier may be fit in block 407; alternatively, if the refit flag is determined to be false, the probability of a thought being generated by the individual may be determined (block 409).

Process 400 may be initialized with one or more pre-existing classifiers configured to produce predictions for the initial class labels.

After the classifier has been fit (e.g., the geometric mean of each thought has been calculated based on a plurality of features), a new weight vector may be calculated having a probability or fidelity rating for each fitted classifier (block 408). When it is the case that a predetermined amount of classifiers are not available, the individual 101 completes a training session, described in detail by process 800, to fit a real-time classifier. Where the training was not initialized with any classifiers, the weight vector has only one fidelity rating and the weight vector is not calculated. When a classifier or classifiers are available during training (e.g., when available at the start of process 400), a weight vector is computed (block 408) and weights are assigned to each classifier associated with the weight vector. Upon subsequent recalculation of the classifier as more features become available (e.g., more events are received associated with the class label for a particular training) to fit the real-time classifier, the weight assigned to the real time classifier will computed by multiplying a predetermined value, generally referred to as a “learning rate”, by the number of features collected (see block 408).

For example, if both an classifier associated with an individual 101 (referred to herein as an “individual classifier”) and a classifier associated with a headset 300 (referred to herein as a “global headset classifier”) are available during the initialization of process 400, or become available during execution thereof, the weight vector has a length of three (corresponding to the individual classifier, the global headset classifier, and the real-time classifier). Based on the number of features associated with each classifier, a weight is assigned to each classifier such that the weight vector sums to 1.0 (e.g., a first classifier may have 300 features, and a second may have 100 features, and the real-time classifier would have no features, each having a weight of 0.75, 0.25, and 0.0, respectively). The weights may be assigned to each classifier in proportion to the features associated with each classifier. In embodiments, the weights may be assigned based on the date and time at which the classifier was generated.

If a learning rate of 0.025 was set for the real-time classifier (e.g., for each identified feature, the real-time classifier increases in weight by 2.5%), when 10 features are available to fit the real time classifier (block 407), the weight assigned to the real-time classifier is 0.25 (or 25%). The remaining weight 0.75 (the total available weight 1.0 minus the learned rate of the real-time classifier 0.25) would be divided between the individual classifier and global headset classifier based on the number of features associated with each classifier. The individual classifier weight would be computed to be 0.525 by multiplying the previously assigned weight of the individual classifier by the remaining percentage of available weight for the weighted vector (0.7×0.75). Similarly, the global headset classifier weight would be computed to be 0.225 (0.3×0.75). As a result of this redistribution or reallocation of weights, the real-time classifier slowly gains influence as the fidelity of the real-time classifier improves (block 412) while still maintaining the data associated with the prior iterations of process 400. The process of slowly increasing the influence of the real-time classifier, in the field of BCI and machine learning, may be referred to as a calibration or a smart initialization.

After fitting and/or refitting the classifier, if the predicting flag is set to false, then process 400 may end or return to block 401 to wait to receive a new sample. Alternatively, if the prediction flag is determined to be set as true (block 409) a prediction counter is incremented (block 410). The prediction counter may be compared to a value “II” (block 411), where value “II” is computed by dividing the sample rate of the biosignal acquisition device 314 (e.g., 250 samples per second) by a predetermined prediction rate (e.g., 10 times a second) to determine a value “II” (e.g., make a prediction every 25 samples). If the prediction counter is determined to be larger than value “II”, then a probability of thought is computed (block 412). Alternatively, when the prediction counter is not greater than value “II”, process 400 waits for an additional sample to be identified at block 401.

When the prediction counter is greater than value “II”, a predetermined duration of the training may be multiplied by the sample rate of the biosignal acquisition device 314 to determine the number of samples to extract from the filtered signal ring buffer to form an epoch (table 1204, FIG. 12). The epoch may undergo further transformation as previously described, (e.g., a covariance matrix may be determined based on the feature associated with the epoch). For each existing classifier, the feature is input to the classifier prediction function and a prediction is determined as previously described (e.g., the distance from feature to aggregate geometric mean of the active class label). Each probability is multiplied by the scalar weight assigned to the classifier (e.g., summation 1307 in FIG. 13) in the weight vector. The sum of the probability multiplied by the respective classifier weight determine a single probability (e.g., probability 1306 in FIG. 13), along with a system time stamp and active class label, are checked to determine whether context action should be executed (block 413).

A context action or set of executable instructions are assigned to the active class label of the training. If the probability for the active class label is determined to pass the criteria for the context action (e.g., the probability is greater than a predetermined threshold) (block 412), the instructions may be transmitted to the remote device for execution (block 414). If the probability is not sufficient to execute the action, process 400 may reiterate when a new sample is received (block 401). In embodiments, criteria may include identifying a predetermined number of features which satisfy the criteria (e.g., identifying a predetermined number of features which have a probability that a brain switch is present greater than a predetermined probability threshold). In embodiments, the criteria may include identifying a predetermined number of features across multiple epochs.

Additionally, or alternatively, as the probability of identifying a brain switch increases, a display output to the individual 101 may be modified to indicate that the probability has increased, commonly referred to as “neurofeedback” (see FIGS. 11A and 11B). For example, in FIG. 11A a graphical user interface or display 1101 is illustrated instructing an individual to “Imagine Closing Left Hand” via instruction 1102 and is also displaying a probability of 0.2 via neurofeedbacks 1103. As the individual begins to imagine the hand movement, all existing classifiers are predicating and being combined to produce a probability that may begin to increase and approach 1.0. Drawn in FIG. 11B is a later point in time where the latest probability of the individual 101 thinking the thought type is 0.95. The diameter of the circle for neurofeedback 1103 is drawn smaller in FIG. 11A than that of the diameter of the circle drawn in FIG. 11B. Neurofeedback 1103 may be very helpful for an individual 101 that is a novice who may need help learning to evoke a thought, this way classifiers from other individuals may assist by providing a visual indication to the individual that they are evoking the correct thought. Another example may be seen in FIG. 1 where the individual may have been verbally instructed to “imagine closing their left hand” and the box on the left is positioned at 0.8 percent of the way up as a way to visually indicate the proper recognition of a brain switch.

FIG. 5 shows a method for determining whether a particular brain switch exists based on comparison of samples compared to one or more previously-fit classifier, and if desired, refit the weight vector based on the samples being analyzed, the method referred to in the art as transfer learning and herein referred to generally as process 500. In many respects, process 500 is similar to process 400, and corresponding descriptions will not be described in detail so as to not obscure the description of process 500.

Initially a new discrete sample is received from the biosignal acquisition device 314 by the computer 309 (block 501). A prediction counter, filter counter, and refit counter may be initialized, similar to the initialization of process 400. A plurality of classifiers (e.g., individual classifiers, global headset classifiers, classifiers stored remotely in database 102, etc.) may be identified, their corresponding weights added to a weight vector initialized at the start of process 500. Each time the weight vector is initialized the weight vector may be filled with values associated with each classifier, the values summing to a total value of 1.0 (e.g., 100%). The weights may be assigned proportionally, in proportion to the number of features which were used to fit the classifier, the age of each classifier, etc. As a result, the classifiers of any individual 101 may be prioritized over those of another individual 101 or subset of individuals 101, enabling the individual 101 to retain classifiers suited to their particular profile. The weight vector may be reinitialized (see block 511) and the weights recalculated upon successive iterations of process 500.

Initially, upon receipt of samples from the biosignal acquisition device 314, the filter counter is incremented. The samples may be added to a working ring buffer of size value “I”, value “I” is calculated by multiplying a predetermined amount of time by the biosignal acquisition device 314 sample rate (e.g., every 0.05 seconds filter the latest samples and add them to a filtered ring buffer), which contains raw samples (e.g., unfiltered samples). If the filter counter is greater than value “I” (a filter threshold), then the samples may be filtered (block 503) and inserted into a filtered signal ring buffer of equal length to the raw sample ring buffer. Once filtered, the counter is reset.

As noted above with respect to process 400, the raw samples in the ring buffer may be filtered by any known method, such as by passing the samples through a band-pass filter (block 503). The filter may be stored and recalled for execution of subsequent filtering. For example, when process 500 is called again, the same band-pass filter may be used, thereby eliminate redundant calculations of the appropriate parameters of the band-pass filter (e.g., frequencies to remove from the samples) for the particular class label or thought type.

If a predicting flag is set as true, the prediction counter is incremented (block 505); alternatively, if the prediction flag is set as false, upon execution of block 511 the flag may be set to true (e.g., enough existing classifiers are initialized). The prediction flag may be set true automatically by an individual 101 (e.g., individual 101 affixes the headset 300 to the top portion of the head of the individual 101 and uses mobile device 103 to evaluate the perceived performance of the transfer learning to determine if the individual 101 should set the predicting flag back to false and initiate a process 800 (see FIG. 8) to fit a new classifier in a process 400).

When the prediction flag is set as true, the prediction counter is incremented (block 505) and the value of the prediction counter is compared to a second threshold (“II”) (block 506). The second threshold may be computed by multiplying a predetermined amount of seconds by the sample rate to determine what may be referred to as a prediction rate (e.g., the number of times a prediction is made every second). When the prediction counter is determined to be larger than the second threshold “II”, the probability of a thought existing in is determined (block 507). If the prediction counter is not greater than the second threshold “II” the refit counter is compared to a third threshold (block 510).

An epoch may be identified from among the plurality of samples and, based on the identification, a feature may be formed according to a predetermined set of mathematical conversions determined by the active class label (see block 405, FIG. 4). Each classifier computes the probability that the electrical activity of the brain indicates that a brain switch is being generated based on the active class label and the identified feature. Each prediction is multiplied by the weight of the classifier stored in the weight vector. The predictions of each classifier are summed together to form a final probability (see example in FIG. 13) that a brain switch is being generated. When the prediction is greater than a predetermined threshold or otherwise satisfies predetermined matching criteria (block 508), a control signal is generated and/or transmitted to a computing device (e.g., mobile device 103) (block 509).

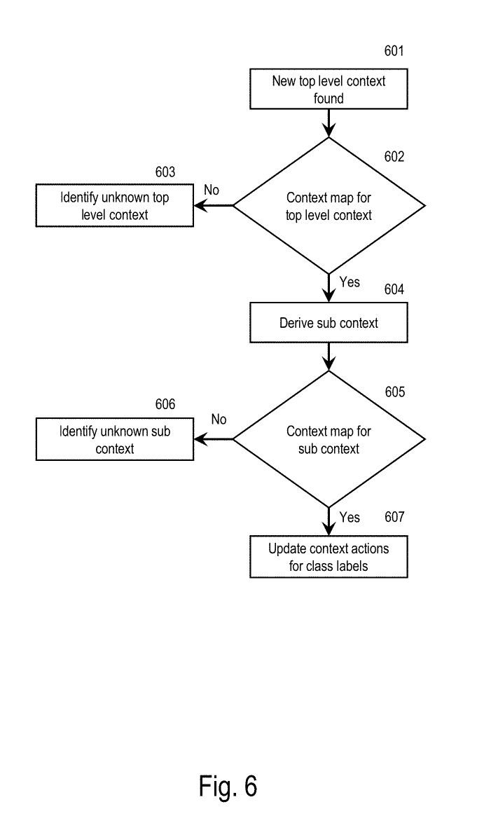

The instructions correspond to a context action which is part of a context map (see process 600, FIG. 6). The criteria used to determine the probability that a brain switch is being generated by an individual may be any criteria known in the art, as well as criteria described above with respect to FIG. 4. If the probability is not sufficient to execute the action, the refit counter is compared to a third value “III” to determine whether it is desirable to recalculate the classifier weights stored in the weight vector (block 510).

Regardless of whether any of the conditions at blocks 504, 506, and 506 are satisfied, the refit counter is compared to the third predetermined threshold, value “III”. If greater than the predetermined threshold, the classifier weights stored in the weighted vector are recalculated (block 511) based on the fitting or refitting of one or more classifiers. If the refit counter is less or equal to the third threshold value “III”, process 500 waits for a new sample to process (block 501).

As noted above, the weights for all initialized classifiers may be recalculated (block 511). For example, as described by Waytowich et. al (Front Neurosci. 2016; 10:430) in a paper called “Spectral Transfer Learning with Information Geometry for a User-Independent Brain Computer Interface,” the contents of which are hereby incorporated by reference in their entirety and are submitted concurrently herewith, the method of applying spectral meta learning can be used to calculate a weight vector for each independently trained classifier. The method may be performed by storing a predetermined number of features most recently determined or identified (e.g., predicting 10 times a second produces three-hundred features over a thirty second period) and computing a “hard” prediction for each classifier. A “hard” prediction or binary prediction refers to using a fit classifier (e.g., a classifier that was previously fit with features for both class label “0” (e.g., rest), and class label “2” (e.g., imagine left hand close)) to internally compute the probability of a feature being a thought type. For example, a classifier that produces a probability of 0.3 (or 30%) for a feature associated with the active class label “2” will determine a hard prediction of 0 (e.g., that a feature with the class label 2 was more likely not present by virtue of being less than 30% certain), whereas if the active class label prediction was 0.8 (or 80%), the hard prediction would be 2.

A hard prediction is computed for every existing classifier (e.g., process 500 was initialized with 200 classifiers) on every feature (e.g., all 300 features created in the past 30 seconds) and stored to a “hard prediction” table. A covariance matrix is then estimated on the hard prediction table (e.g., the covariance of a 200 classifiers by 300 features matrix will form a 200 by 200 square matrix of covariances). The resulting covariance matrix compares the similarity in individual hard predictions between each classifier over predetermined length of time (e.g., over the 30 seconds). Lastly, a general eigenvalue problem is computed on the square covariance matrix to determine a principal eigenvector. The resulting principal eigenvector reveals which classifiers are the most similar and may be used to update the classifier weights in the weight vector. For a detailed description of the eigenvalue problem and principal eigenvector, reference may be made to U.S. Pat. No. 6,944,602 entitled “SPECTRAL KERNELS FOR LEARNING MACHINES,” the contents of which are hereby incorporated by reference in their entirety. The new weights in the principal eigenvector may be further optimized through an iterative estimation maximization process as described in Parisi et. al (Ranking and combining multiple predictors without labeled data. Proc. Natl. Acad. Sci. U.S.A. 111, 1253-1258, available at www.pnas.org/content/111/4/1253, the contents of which are hereby incorporated by reference in their entirety, and submitted concurrently herewith. The new weight vector is used when subsequent probabilities of the generation of a thought associated with a brain switch is calculated (block 507).

FIG. 6 shows a method for deriving what an individual 101 may be focused on in accordance with embodiments of the present disclosure. A context detection system may perform certain functions of the method, which is generally referred to as process 600. Iterations of process 600 may be executed to provide context maps for execution of context actions during execution of processes 400 and/or 500.

A context map, either a default or global map or map associated with an individual, may include one or more top-level contexts and one or more sub-contexts, the sub-contexts corresponding to one of the top-level contexts. As used herein, the term “context map” is an indexed map having both top-level contexts and sub-contexts associated with one or more class labels. Context maps may be associated with an individual 101, a group of individuals 101, or may be global (e.g., available to all computing devices). In embodiments, the context map may only include top-level contexts associated with a particular set of instructions to be executed based on the detected generation of a brain switch. Additionally, or alternatively, the context map may have many sub-contexts associated with a particular brain switch.

In embodiments, the context maps may be set by the application or device receiving the context commands. More specifically, as an individual 101 interacts with a computing device 200 (e.g., mobile device 103), the context map may switch the control signals executed when a brain switch is generated. For example, as an individual 101 generates thoughts of closing their right hand (a first brain switch) the computer 309 may sense the brain switch and, in accordance with a context command (or instructions) of a first context map, cause a first control signal to be transmitted to the mobile device 103 to control the mobile device 103 (e.g., to increase the volume). As the individual engages with the mobile device 103 and changes the state of the mobile device 103 (e.g., switches to a different application) a second context map may be loaded and, as the brain switch (closing the right hand of the individual 101) is generated, a second context command may be transmitted to the mobile device to control the mobile device 103 in the changed state (e.g., to pause the playback of a video).

For example, an individual 101 may be using an internet browser (a top-level context) and may be on a particular website (e.g., news website, sub-context level “1”) and may be viewing a particular page within the particular website (e.g., breaking news, sub-context level “2”) performing a particular action (e.g., watching a video, sub-context level “3”) that may have a plurality of associated thought types (class labels) associated with executable actions (e.g., imagining the closing of the left hand pauses the video, imagining the closing the right hand mutes the video, and imagining the lifting a big toe scrolls down on the web page).

When context maps have been loaded into the memory 204 on the computer 309, the top-level context identified (block 601) may be compared against top-level contexts for each context map. If the top-level context is matched with a context map loaded in memory, the sub-contexts may be identified (block 604). If no corresponding context map is found, then the unknown top-level context is identified (block 603).

The unknown top-level context may be stored in the memory 204 of the computer 309, sent to a database 102, and discarded (block 603). In embodiments, additional context maps may be loaded into the memory of the computer 309. In embodiments, the additional context maps may be loaded from remote computing devices in electrical communication with the computer 309 (e.g., database 102).

Where no top-level context is identified, the computer 309 may generate one or more top-level contexts. Top-level contexts may be generated by identifying interactions between the individual 101 and a computing device (e.g., mobile device 103) controlled by the individual 101 to determine what commands are commonly executed. For example, if an individual 101 executes a command to start a camera on the mobile device 103 frequently, the command may be identified as a top-level context.

One or more sub-contexts may be derived based on the identified top-level context (block 604) For example, a top-level context may be to open or execute a web browser and the sub context may be to cause the web browser to navigate to a predetermined webpage. Similarly, a context may be to unlock a Bluetooth-controlled lock, and the sub context may be to advance (e.g., physically move closer) a wheelchair towards the lock.

If a context map is found which matches the top-level context (block 602) and matched to a derived sub-context (block 604) based on an initial top-level context (block 601), then the context map may be used to update the associated class labels with the context actions (block 607). If the sub context was not able to be derived, then the unknown sub context may be added to the memory of the computer 309, sent to the database 102, or ignored (block 606). The individual 101 controlling the computer 309 (e.g., the individual 101, a remote service/content provider, etc.) may be able to add context maps having sub-contexts.

The context map (either generated or pre-existing) may be used to update the active context actions for processes 400 and 500 (executed at blocks 414 and 509, respectively). A context map may have a plurality of context actions, each context action being associated with a class label. When processes 400 and 500 call a context action in response to determining a brain switch is being generated, the brain switch associated with the context action, the context action may be executed on computer 309 (e.g., turn pause music playing on headwear 350) or transmitted from the computer 309 to a computing device (e.g., computer 309, a remote computing device (not explicitly shown), etc.) for execution on a processor 202 thereof.

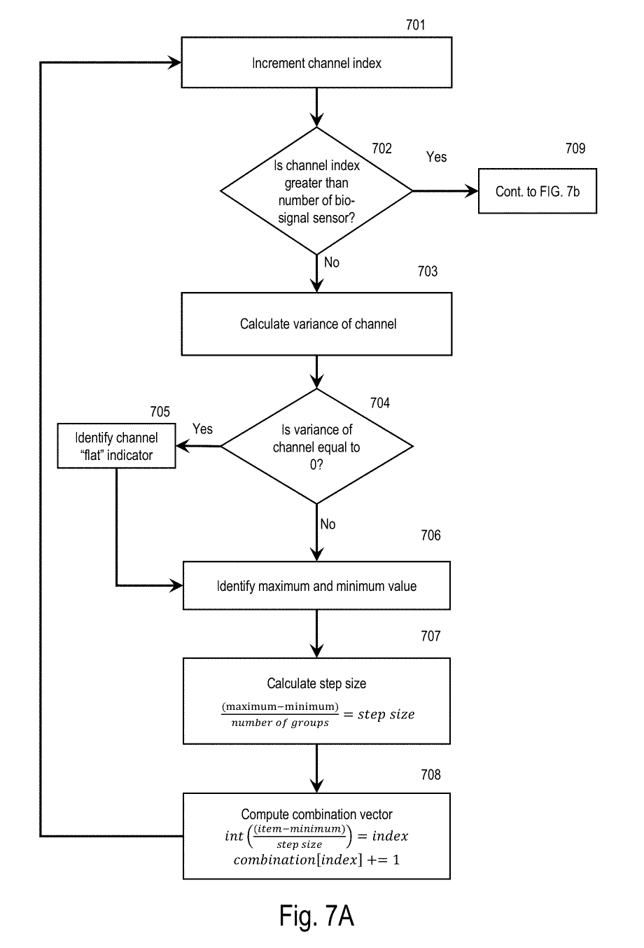

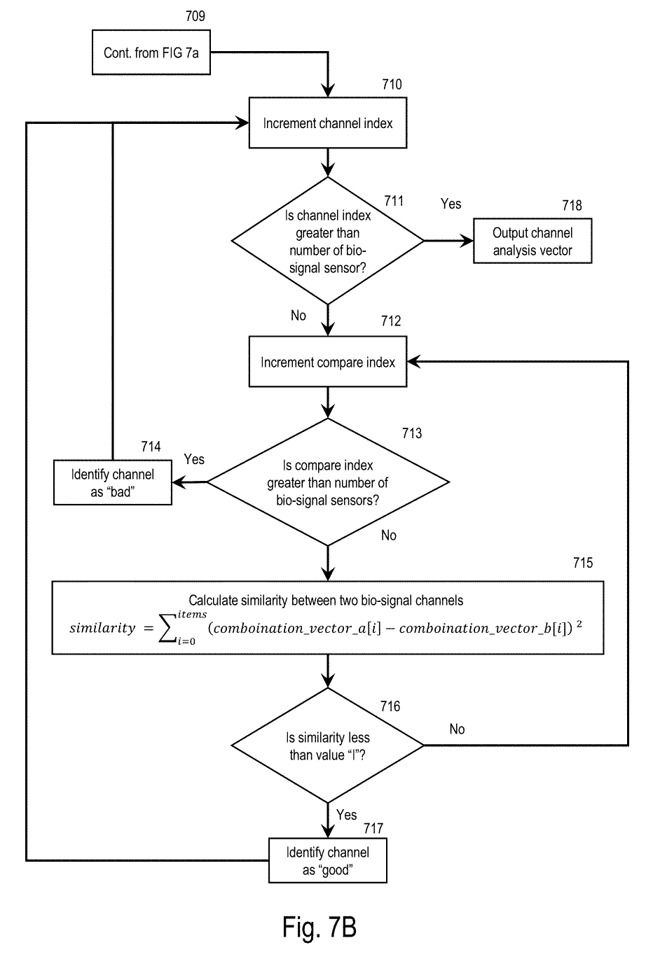

Referring now to FIGS. 7A and 7B, illustrated is a method for determining the signal quality of multiple biosignals in real-time, the method generally referred to as channel analysis or process 700. The overall idea behind process 700 is that when two electrodes that are physically close to each other (e.g., an inch apart) are in good contact with the skin of an individual, the biosignals recorded are very similar to each other. In the art, EEG professionals are trained to read real time graphs of EEG and recognize “good” EEG signals by when they are all very similar. Process 700 algorithmically performs the same type of identification such that an individual 101 that is a novice does not have to learn how to read real-time EEG plots to determine when an electrode is collecting “good enough EEG”. Without process 700, performance of process 400 may be severely impacted and process 500 may not be possible because flat or bad electrodes can cause calculation errors on classifiers when they are fit or predict on features.

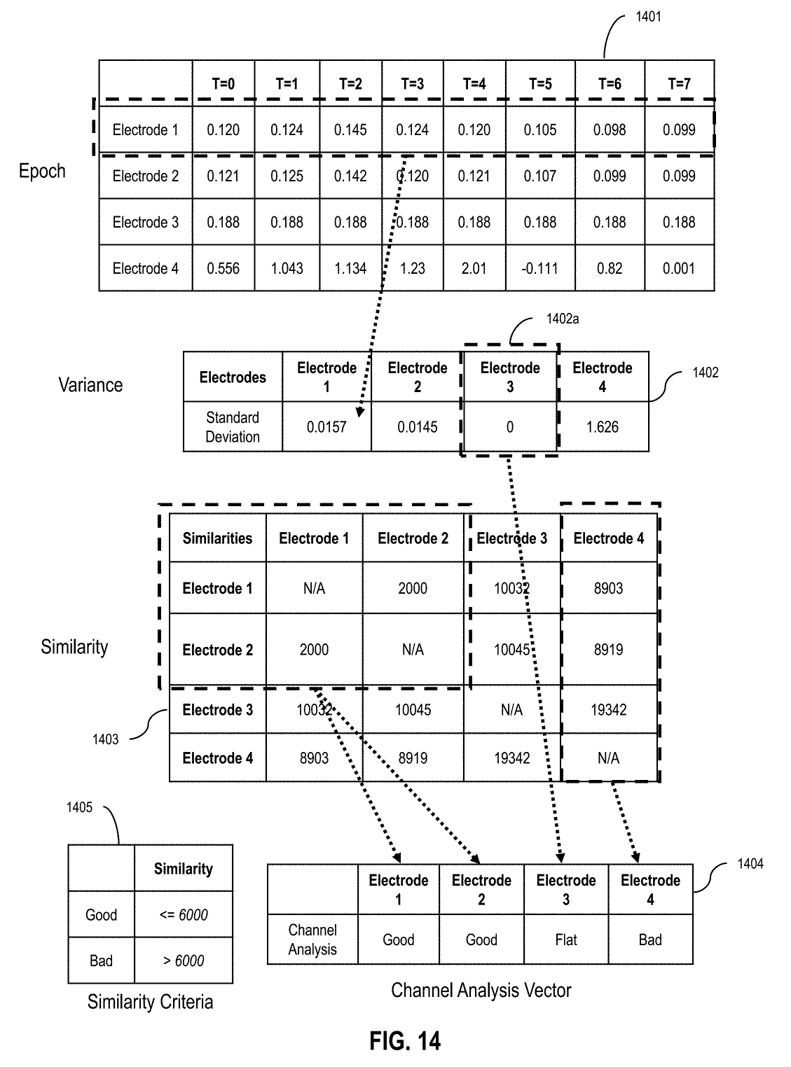

As samples are received by the biosignal acquisition device 314 and are transmitted to the computer 309, the samples are divided into epochs (see table 1401, FIG. 14) As used herein, the term “channel” denotes all data in an epoch received and/or otherwise associated with a particular biosensor 320 (e.g., an EEG electrode; biosensor 320, FIG. 3B, etc.).

Initially, a channel analysis vector and channel index may be initialized, the channel analysis vector having a length equal to the amount of biosensors 320 configured to communicate with, but may not necessarily be in electrical communication with, the biosignal acquisition device 314.

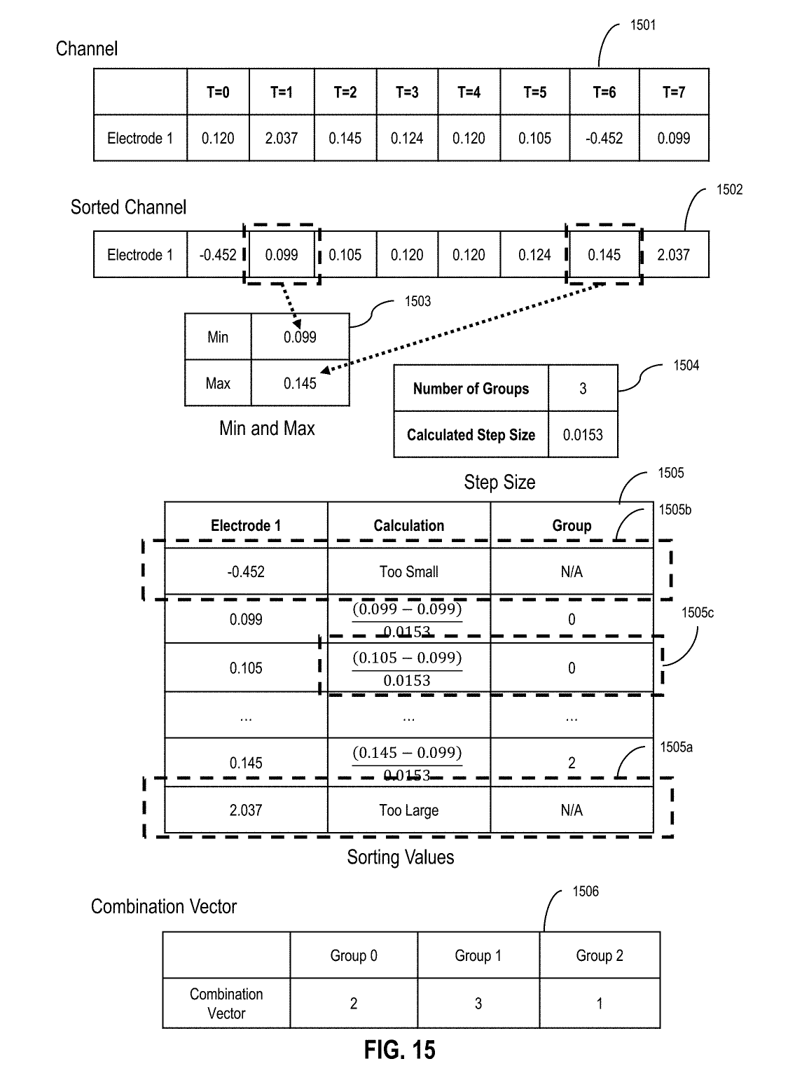

If the length of the channel index is determined to be greater than the number of biosensor 320 expected to be in electrical communication with the biosignal acquisition device 314 (block 702), the “combination vectors” (see block 708; table 1506 in FIG. 15) associated with each channel are computed and process 700 continues to block 709. If the channel index is less than or equal to the number of biosignal sensors (or channels), process 700 continues to block 703 where a variance is calculated. As described here, the combination vector includes identifiers associated with groups in which the values for a particular biosensor 320 correspond (see FIG. 15).

A variance for a channel is calculated in block 703. As used herein, “variance” is the standard deviation of all voltages in the channel squared. When the variance of the channel is determined to be equal to 0, or to be within a predetermined threshold of 0 (block 704), the channel is identified as being flat (block 705). As used herein, a “flat” channel denotes a biosignal sensor that may be disconnected from the scalp or other portion of the body of the person or individual 101 (e.g., there is an air gap between electrode and skin), that the biosignal acquisition device 314 input may be broken, or the electromechanical connection from biosensor 320 via wire 313 is otherwise inoperable. A flat channel, and more particularly samples received from the flat channel, are removed prior to execution of or throughout the process 400 and the classifier is refit (see block 407 without the samples received from the flat channel. Alternatively, if the channel is not determined to be flat (block 704), process 700 continues and calculates the combination vector for each vector (block 708). For an example of a flat channel, see table column 1402a (FIG. 14) where the variance of “Electrode 3” is equal to zero (e.g., each sample is the same voltage for the entire epoch), the corresponding channel analysis vector in table 1404 associated with “Electrode 3” is marked as “flat”. In addition to being determined to be flat or not flat, a channel may be determined to be deficient (e.g., the samples associated with the channel are indicative of a poor connection between the biosensor 320 and the scalp of the individual 101. Similar to flat channels, signals received from channels identified as deficient may also be removed prior to the execution of, or during execution of, process 400. Once removed, the remaining samples associated with the channels that are not determined to be flat or deficient may be used to fit one or more classifiers (process 400).

Process 700 continues to compute a combination vector, by initially identifying minimum and maximum values (see table 1503, FIG. 15) of the samples received for the channel being analyzed. Any particular channel (e.g., “electrode 1” at table 1501, FIG. 15), may be sorted such that the samples are placed in ascending order (see table 1502, FIG. 15). The step size may be computed (block 707). The minimum value among the sample values may be subtracted from the maximum value among the samples and then divided by a predetermined value, referred to as number of groups (see table 1504, FIG. 15). The channel may be sorted in block 708 by the step size.

When computing a combination vector for a particular channel, the combination vector may be initialized. For each element in the channel, where each sample received by the computer 309 from the biosensor 320 (e.g., each voltage reading) is compared to determine if the sample value is greater than or equal to the maximum value computed at block 706. If the sample is greater than or equal to the maximum value, this item is excluded from the combination vector (see table 1505a, FIG. 15) and the next sample in the combination vector is checked. The sample may also be checked to see if the value of the sample is less than the minimum value computed in block 706. If the item is less than the minimum value, the item is excluded from the combination vector (see table 1505b, FIG. 15). The counter to increment's index may be calculated by first subtracting the minimum value from the item then dividing by the step size computed in block 707 and finally truncating the result by removing anything in the decimal places (see an example of the calculation in table 1505c, FIG. 15; the equation may be seen on block 708). The value at the index within the combination vector may be incremented (block 708).

When it is determined that the channel index is greater than the number of available channels (block 702), the channel index is set to −1 such that it may be reused by the remainder of process 700 and may be incremented to 0 in block 710.

In block 711, where if the channel index is greater than the number of biosignal sensors the channel analysis vector is output (block 718). If the channel index is less than or equal to the number of biosignal sensors, a compare index is initialized to 0 (block 712) and the similarity between the channel at channel index and the channel at the compare index is determined.

When it is determined that the compare index is greater than the number of biosignal sensors (block 713) the channel associated with the channel index may be identified as “bad” (block 714). If the compare index is less than or equal to the number of biosignal sensors, the similarity between two predetermined channels is determined (block 715).

Two channels may be evaluated to determine the similarity between the two channels by comparing their respective combination vectors (block 715). The first combination vector may be the combination vector for the combination stored at the index of the channel index and the other combination vector may be combination stored at the compare index. A similarity value may be computed between two combination vectors via an elementwise subtraction of one combination vector from the other and then the differences are squared and summed together. If the value corresponding to the similarity of the channels is determined to be less than the value “I” (block 716), the channel is identified as functional or good (block 717). Alternatively if the similarity of the channels is determined to be greater than or equal to the value “I”, process 700 continues to block 712.

When the channel at the channel index and/or compare index in the channel analysis vector is identified at “good” process 700 continues to block 710. The indication may also contain a metric of quality computed by the division of the similarity value by the value “I”.

When the channel analysis vector is analyzed (e.g., the channel index is greater than the number of biosignal sensors of the system) the channel analysis vector is stored in the memory 204 of the computer 309 for use by processes 400 and/or 500 (block 718) or sent to a database 102 for the use in analytics. The channel analysis vector may be used by multiple processes such as, without limitation, a graphical user interface outputting an illustration indicating the quality of each electrode, an instance of process 400 and/or process 500 for the elimination of flat or bad channel from calculation, and the like.

FIG. 8 shows a method for improved training of an individual 101 to differentiate between brain switches or thoughts, referred to generally as process 800. The method may be manually implemented by the user to learn a thought. Process 800 is initialized with a list of class labels and a number of trials to run or value “I”. The stimulus counter is initialized to 0. Active artifact detection may be used to save the time an artifact was detected and may prohibit the process 800 from identifying a stimulus or creating an event for process 400. Channel analysis, or process 700, may be performed at the start of each process 800 to verify the quality of each biosensor, where a flat channel may be removed and avoided in block 405 computing the feature matrix for process 400. A unique identification may be assigned to the process 800. Time synchronization between the headset 300 described above and other external computing devices may be critical for time bound thought decoding, for illustrative purposes, the energy potential (P300) evoked potential that is elicited when a stimulus is identified with an individual 101 with a photo of famous person such as Albert Einstein. The stimulus offsets must be aligned with the closest biosignal sample. Biosensor time sync may be imperative to forming epochs which are transformed and piped into classifier fitting in process 400. Every process may sync to the same headset system time. A translation of headset system time and the external process system time may need to be found. A transmission system from and external process may attempt to measure and find an average amount of time for a packet to be sent to and from the headset.

A method of training one or more classifiers, generally referred to as process 800, starts by initializing a trial (block 801). Upon each iteration of process 800, an individual is caused to generate brain switches to produce samples, the samples aggregated into events for use by process 400 (see block 404). A training session may be initialized with one or more thought prompts to for fitting multiple classifiers for each thought or classifiers that may predict a context command from multiple thoughts. The training may show the same thought prompt a predetermined number of times, to improve the accuracy of classifiers built during the execution of process 400 (at block 406). The training may alternate between projecting differing thought prompts to the individual 101 to clear the mind of the individual 101 and encourage them to stop generating signals associated with the prompted brain switch.

A stimulus vector is initialized (block 801). The stimulus vector may be initialized as having the length of a predetermined number of stimulus to be output to the individual during a training (“value ‘I’”). The stimulus may be any visual, audible, or other suitable sensory output displayed or otherwise output to the individual 101 via the mobile device 103 or by any other remote computing device having a display or other suitable output interface configured to evoke a specific thought as well as a baseline thought or thoughts (brain switches). Each element of the stimulus vector will contain a specific thought, or a baseline thought type. A stimulus counter may be initialized to be incremented each time the stimulus associated with the stimulus vector element is presented to the individual 101 (block 802).

As the stimulus counter is incremented (block 802), the stimulus counter is compared to a first threshold “I” (block 803). If the stimulus counter is less than or equal to the predetermined value “I”, the stimulus is presented to the individual 101. If the stimulus counter is greater than value “I”, it is determined whether another trial is desired (e.g., the individual 101 may be prompted as to whether they would like to receive an additional stimulus) (block 810). If another training is desired, the stimulus counter is re-initialized (block 811). Alternatively, if another training is not desired, the data from the training is saved to the memory 204 of the computer 309, database 102, and/or a remote computing device (block 812). As used herein, the term “data” refers to biosignal samples, epochs, features, class labels, unique identifiers, and classifiers from process 400 and any other information recorded during the trial such as tags for events such that epochs and features may be extracted from the biosignal samples at a later time (e.g., a new method for going from epochs to features and fitting a classifier is implemented and the database 102 runs a process 400 using the new methods and producing improved classifiers that may be utilized by either process 400 and/or process 500 running on headset 300). The data may be stored locally to the headset memory, to a database 102, or to a computing device running a training application.

When the stimulus counter is determined to be less than or equal to the stimulus threshold value “I” (block 803), the stimulus may be presented for identification to the individual 101 (block 804), to provoke the individual 101 to generate a specific brain switch or thought to be sampled by the headset 300. The stimulus counter may be used to indicate what stimulus to present to the individual 101 to evoke a specific thought (e.g., when cycling through a plurality of stimuli during a training of multiple classifiers). For example, as shown in FIGS. 11A and 11B a prompt is shown to the individual 101. The stimulus may be selected and/or determined by the individual (e.g., the individual 101 may input text to cause themselves to generate a brain switch). Additionally, or alternatively, the stimulus may be stored among a plurality of pre-generated stimuli.

If the stimulus is dependent on another process (e.g., a process running on the computer 309, mobile device 103, etc. which manages the drawing of an image on the display of the device), the time associated with the event created may be synchronized with the headset 300 system time associated with the time at which the screen is redrawn. The synchronization may be performed to correct for any temporal offsets between when the command to display the stimulus and the actual time at which the stimulus was presented and removed from the view of the individual 101. If the active stimulus is not dependent on another process, (e.g., an audio tune is played on headwear 350) the time used to create the event for the stimulus may use the headset 300 system time.

There may be a plurality of different stimulus for each class label that results in a higher than normal probability of detecting a thought from a plurality of samples. For example, the stimulus for one iteration of block 804 may be a visual instruction on a page (see FIG. 11A) whereas the next stimulus for block 804 for the same thought type (class label) may be an audio sound that the individual 101 was instructed imagine the specified thought type upon hearing. To facilitate bagging, a process described previously, block 804 may set a plurality of timers to create new events near the end of the stimulus presentation (block 805).

While the stimulus is being presented, the headset 300 receives samples and stores the samples in the memory 204 of the computer 309 as an event. The event is associated with the active stimulus, the event having a duration extending across a predetermined time period or across the entire time samples were received while presenting the stimulus (block 805). The event may further be associated with a time stamp or plurality of time stamps for each sample or subset thereof, a class label and a refit status flag. The refit flag may be used to trigger a process 400 to fit and/or refit a classifier.

The stimulus presented may be removed (e.g., display and/or audible output associated with the stimulus may be discontinued) (block 806). The stimulus may be discontinued once a predetermined period of time has expired during which the stimulus was presented to the individual 101. Once discontinued, a baseline or control stimulus may be displayed or otherwise presented to the individual (block 807). The baseline stimulus may be any stimulus configured to invoke a brain switch different from the brain switch invoked by the stimulus presented at block 804. For example, FIGS. 11C and 11D show examples of stimuli or instruction 1102 such as text which prompts the individual 101 to “remain still for the artifact detector scan,” or to “relax.” It is contemplated that a control stimulus may be any visual or audible stimulus, such as, without limitation, the presentation of an image of a duck, a series of audible tones or beeps, etc. A plurality of control stimuli may be presented in a random order to prevent the individual 101 for preparing for the control stimulus.

As the control stimulus is presented to the individual 101 (block 807) the computer 309 may receive a plurality of samples from the biosignal acquisition device 314 and associate the samples with events. Each event may include information such as a duration (either the length of the presentation of the stimulus or a subset of such length), a time stamp, a class label and a refit status flag. The event is stored in the memory 204 of the computer 309 (or a remote computing device such as database 102). Once the event or events are created, the control stimulus may no longer be presented to the individual 101 (block 809) and process 800 may return to block 802 (block 809).

Referring to FIGS. 9A and 9B, an electrode 900 is illustrated according to aspects of the present disclosure. The electrode may be made of various materials which at least partially encase conductive components therein (described below). It is contemplated that the electrode 900, and more particularly the electrode housing, may be formed of a non-conductive polymer silicone rubber, or any suitable biocompatible material known in the art.

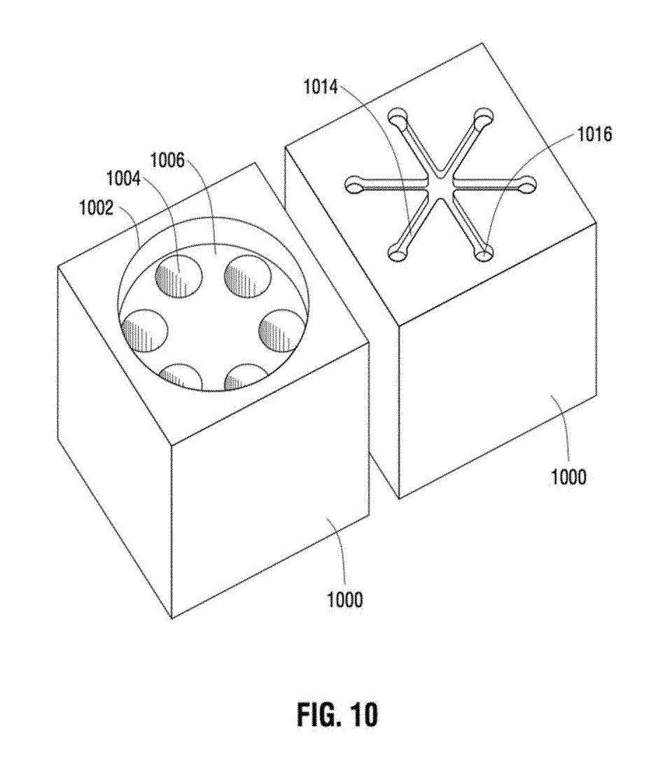

Connectors 905 are operably coupled electrically by wires 901 to prong tips 904, respectively. The prong tips 904 are disposed on arms or prongs 903 extending from a bottom portion 902a of the electrode housing 902. The wires 901 may be physically connected via an electromechanical connection such as a soldered connection. In embodiments, the connectors 905 and wires 901 may be cast into a mold (FIG. 10).

The electrode housing 902 comprises a biocompatible substrate forming the top portion 902b and a bottom portion 902a. More particularly, the electrode housing 902 is formed of a non-conductive material such as silicone. In embodiments, the electrode housing 902 may be formed of a material configured to reduce interference, e.g., a metal configured to shield the electrode from environmental noise. The bottom portion 902a is configured to face or otherwise be oriented toward the scalp of an individual 101 (FIG. 1) when in contact with the scalp of the individual 101. The top portion 902b of the electrode housing 902 is configured to operably couple to a wire or wires to transmit signals therefrom. For example, the electrode 900 may be used in connection with the biosignal acquisition device 314 (FIG. 3A) to transmit signals measured and associated with activity in the brain of the individual 101.

The prongs 903 extend from the bottom portion 902a of the electrode housing 902 and have a wire 901 disposed along a bore extending through the prong therein. The prong 903 may be configured to engage the scalp of the individual 101 based on the length of the hair on the scalp of the individual 101. For example, an individual 101 who has hair extending an inch from the scalp of the individual 101 may use prongs having a greater length than the prongs used for an individual 101 having hair extending a quarter of an inch from the scalp. In embodiments, the prongs 903 may be removably disposed to the bottom portion 902a of the electrode housing 902. It is contemplated that the length of the prongs 903 may be anywhere from 1 mm to 50 mm in length.

A prong tip 904 is coupled to the distal portion of the wire 901 extending through the prong 903. The prong tip 904 may be made of a material different than wire 901, the material comprising an ion-to-electron charge carrying conversion desirable for receiving and transferring bio-signals to the biosignal acquisition device 314. Prong tip 904 may also be made of a bio-safe material that will not cause medical problems for an individual. While shown as having a rounded tip, the prong tip 904 may be any suitable shape configured to enable electrical contact with the scalp of the individual 101.

The connector 905, may be any rigid or semi-rigid material that which is electrically conductive. The connector 905 may have a mounting surface configured to couple to a connector (e.g., connector 319, FIG. 3B). The connector 905 may be configured to removably connect to the connector 319 (e.g., via a threaded connection between the connector 905 and the connector 319, to enable exchange of an electrode 900 when desired. The threaded portion of the connector 905 may be made of a conductive material.

Referring now to FIG. 10, an electrode mold 1000 is illustrated according to aspects of the present disclosure. The electrode mold 1000 may be formed of any rigid or semi-rigid material capable of receiving material to form the electrode 900 of FIG. 9. The electrode mold 1000 may be made of a rigid polymer or metal (for injection molding), the material configured to receive pliable substrates used to form the electrode 900 during manufacturing.

A bottom portion 1006 forms a recess extending from a top portion of the electrode mold 1000, the top portion of the electrode mold 1000 corresponding to the bottom portion of the electrode 900. The recess extending through the bottom portion 1006 defines an aperture 1002 having a diameter equal to a diameter of the electrode base 902. A plurality of prong cavities 1004 extend downward from a bottom portion 1006 of the recess of extending downward from the top portion of the electrode mold 1000. More particularly, the prong cavities 1004 extend downward and outward and mate with a plurality of openings 1016 disposed along a bottom portion of the electrode mold 1000.

A flex charge carrier channel 1014 provides a channel for wire 901 to rest in and against while raw materials are poured or otherwise disposed in the electrode mold 1000 as an electrode housing solidifies. The opening 1016 may be configured to receive the wire 901 of an electrode 900 as the electrode housing is formed. In embodiments, the wire 901 may be tied off or otherwise maintained away from the openings 1016 to maintain the position of the wire 901 relative to the electrode mold 1000 during manufacture of an electrode 900.

As described above, FIGS. 11A-11D illustrate varying stimuli configured to be displayed on a display of a computing device (e.g., the display of the mobile device 103). More particularly, displays 1101 illustrate stimuli configured to cause an individual 101 to generate brain switches associated with a specific thought or specific thoughts. Displays 1101 have instructions 1102, respectively, disposed thereon in the form of images. The images are configured to prompt the individual 101 to generate certain brain switches. In embodiments, video, text, audio clips or blank screens may be displayed or otherwise output to invoke the desired brain switch in the individual 101.

While several embodiments of the present disclosure are shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed is:

1. A biosignal acquisition system for transmitting context commands based on sensed biosignals, the system comprising:
a computer;
a biosensor configured to be in contact with a scalp of an individual; and
a biosignal acquisition device operably coupled to the biosensor and configured to amplify signals received by the biosensor and communicate the amplified signal to the computer,
the computer comprising a processor and memory having instructions which, when executed by the processor, cause the computer to:
receive the amplified signals from the biosignal acquisition device,
generate a feature matrix based on the amplified signals,
identify a brain switch associated with each feature in the feature matrix,
predict a probability that a classifier is associated with the brain switch of each feature, and
fit the classifier based on the feature matrix.

2. The system of claim 1, wherein the memory has further instructions which, when executed by the processor, cause the computer to:
initialize a weight vector having a weight associated with the classifier, the weight corresponding to a probability that the classifier is reliable.

3. The system of claim 2, wherein additional signals are received from the biosensors and amplified by the biosignal acquisition device prior to being transmitted to the computer.

4. The system of claim 3, wherein the memory has further instructions which, when executed by the processor, cause the computer to:
generate a second feature matrix based on the additional amplified signals;
identify the brain switch associated with each feature of the second feature matrix;
predict the probability that a real-time classifier is associated with the brain switch of each feature;
fit the real-time classifier based on the second feature matrix; and
refit the classifier based on the fitting of the real-time classifier.

5. The system of claim 4, wherein refitting the classifier includes adding a weight of the real-time classifier to the weight vector.

6. The system of claim 5, wherein the weight of the real-time classifier stored in the weight vector is increased as additional amplified samples are received.

7. The system of claim 6, wherein the memory has further instructions which, when executed by the processor, cause the computer to transmit a control signal based on the prediction in a case where the prediction satisfies prediction criteria.

8. The system of claim 1, wherein the memory has further instructions which, when executed by the processor, cause the computer to:
determine if a subset of signals is associated with a flat channel; and
in a case where the subset of signals is determined to be associated with a flat channel, discard the subset of signals.

9. The system of claim 8, wherein the memory has further instructions which, when executed by the processor, cause the computer to:
initialize a weight vector having a weight associated with the classifier, the weight corresponding to a probability that the classifier is reliable.

10. The system of claim 9, wherein additional signals are amplified by the biosignal acquisition device and transmitted to the computer.

11. The system of claim 10, wherein the memory has further instructions which, when executed by the processor, cause the computer to:
generate a second feature matrix based on the additional amplified signals;
identify the brain switch associated with each feature of the second feature matrix;
predict the probability that a real-time classifier is associated with the brain switch of each feature;
fit the real-time classifier based on the second feature matrix; and
refit the classifier based on the fitting of the real-time classifier.

12. A biosignal acquisition system for identifying brain switches and transmitting context commands based on the brain switches, the system comprising:
a computer;
a biosensor configured to be in contact with a scalp of an individual; and
a biosignal acquisition device operably coupled to the biosensor and configured to amplify signals received by the biosensor and communicate the amplified signal to the computer,
the computer comprising a processor and memory having instructions which, when executed by the processor, cause the computer to:
receive the amplified signals from the biosignal acquisition device,
determine if a subset of signals are associated with a channel having a deficient signal,
in a case where the subset of signals are determined to be associated with a flat channel, discard the subset of samples,
generate a feature matrix based on the remaining amplified signals,
identify a brain switch associated with each feature in the feature matrix,
predict the probability that a classifier is associated with the brain switch of each feature, and
fit the classifier based on the feature matrix.

13. The system of claim 12, wherein the memory has further instructions, which when executed on the processor, cause the computer to determine the quality of the deficient biosensors relative to a set of non-deficient biosensors based on comparing the signals associated with the deficient biosensors and the signals associated with the non-deficient biosensors, and
wherein the set of non-deficient biosensors comprises the biosensors not identified as deficient.

14. A biosignal acquisition system for identifying context commands and controlling a computing device based on the identified context commands, the system comprising:
a computer;
a biosensor configured to be in contact with a scalp of an individual; and
a biosignal acquisition device operably coupled to the biosensor and configured to amplify signals received by the biosensor and communicate the amplified signal to the computer,
the computer comprising a processor and memory having instructions which, when executed by the processor, cause the computer to:
receive the amplified signals from the biosignal acquisition device,
generate a feature matrix based on the amplified signals,
identify a brain switch associated with each feature in the feature matrix,
predict the probability that a classifier is associated with the brain switch of each feature, and
fit the classifier based on the feature matrix, and
a computing device in electrical communication with the computer, the computing having a processor and memory having instructions which, when executed by the processor, cause the computer to:
receive a control signal based on a context map stored in the computer and the prediction performed by the computer, and
perform a function based on the received controls signal.

15. The system of claim 14, wherein the memory of the computing device further has instructions stored thereon which, when executed by the processor of the computing device, cause the computing device to:
transmit a control signal to the computer to swap the context map for a second context map.

16. A method of transmitting context commands based on sensed biosignals, the method comprising:
receiving a first set of signals from an electrode in close proximity to a scalp of an individual;
generating a feature matrix based on the first set of signals;
identifying a brain switch associated with each feature in the feature matrix;
predicting the probability that a classifier is associated with the brain switch associated with each feature; and
fitting the classifier based on the feature matrix.

17. The method of claim 16, further comprising:
initializing a weight vector having a weight associated with the classifier, the weight corresponding to a probability that the classifier is reliable.

18. The method of claim 17, further comprising:
receiving a second set of signals;
generating a second feature matrix based on the second set of signals;
identifying a brain switch associated with each feature of the second feature matrix;
predicting the probability that a real-time classifier is associated with the brain switch of each feature of the second feature matrix;
fitting the real-time classifier based on the second feature matrix; and
refitting the classifier based on the fitting of the real-time classifier.

19. The method of claim 18, further comprising transmitting a control signal based on the prediction in a case where the prediction satisfies prediction criteria.

20. The method of claim 16, further comprising:
determining if a subset of signals is associated with a flat channel; and
in a case where the subset of signals is determined to be associated with a flat channel, discarding the subset of samples.

21. The method of claim 20, further comprising initializing a weight vector having a weight associated with the classifier, the weight corresponding to a probability that the classifier is reliable.

22. A method of transmitting control signals based on sensing one or more brain switches, the method comprising:
receiving amplified signals from a biosignal acquisition device,
generating a feature matrix based on the amplified signals,
identifying a brain switch associated with each feature in the feature matrix,
predicting the probability that a classifier is associated with the brain switch of each feature,
fitting the classifier based on the feature matrix,
transmitting a control signal based on a context map and the predicting, and
performing a function based on the transmitted control signal.
